(19) United States
(12) Patent Application Publication
(10) Pub. No.: US 2025/0317697 A1
(43) Pub. Date: Oct. 9, 2025

(54) LOUDSPEAKER SYSTEM AND HEARING CORRECTION SYSTEM AND METHOD

(71) Applicant: Sound United, LLC, Carlsbad, CA (US)

(72) Inventor: Bradley M. Starobin, Owings Mills, MD (US)

(21) Appl. No.: 19/171,035

(22) Filed: Apr. 4, 2025

Related U.S. Application Data
(60) Provisional application No. 63/575,087, filed on Apr. 5, 2024.

(51) Int. Cl. H04R 25/00

(52) U.S. Cl. CPC H04R 25/505

(57) ABSTRACT

A loudspeaker system and method can measure a specific user's hearing and implement compensatory or corrective processing to address the user's hearing deficiencies. The user's hearing acuity may be determined by (i) headphone techniques in which frequency-based hearing thresholds are determined; (ii) a loudspeaker system set up as intended for use in an acoustic space to emit tonal stimuli; or (iii) inducing, acquiring, and interpreting otoacoustic emissions. Once the hearing profile is determined, appropriate signal processing parameters can be used to generate a corrected audio output signal adjustment. The administered hearing test results can include a plurality of hearing loudness threshold levels at respective test frequencies. The hearing correction system's corrected audio output can be generated from corrected loudness values at selected correction frequency sub-bands.

### CROSS-REFERENCE TO RELATED APPLICATIONS

This non-provisional patent application claims priority under 35 U.S.C. 119(e) to U.S. Provisional Patent Application No. 63/575,087, filed on Apr. 5, 2024, and entitled “LOUDSPEAKER SYSTEM, HEARING CORRECTION SYSTEM AND METHOD,” which is hereby incorporated by reference herein in its entirety.

The subject matter of this application is broadly related to the subject matter of U.S. Pat. No. 9,497,530, 10327064, 10327086, 11838740 and 11900909 (which are incorporated herein by reference for all that they disclose). U.S. patent application Ser. No. 18/903,481, filed Oct. 1, 2024, and titled USER SPECIFIC AUDITORY PROFILES, is hereby incorporated by reference and made part of this specification for all that it discloses. U.S. Patent Application Publication No. 2023/0319478, published Oct. 5, 2023 and titled “LOUDSPEAKER SYSTEMS” is incorporated by reference and made part of this specification for all that it discloses. U.S. patent application Ser. No. 17/933,661, filed Sep. 20, 2022, and titled “SYSTEM AND METHOD FOR ADJUSTING LOUDSPEAKER PERFORMANCE BASED ON LISTENER LOCATION” is incorporated by reference and made part of this specification for all that it discloses.

### BACKGROUND

Embodiments disclosed herein relate to hearing correction systems, products, and methods for implementing compensatory processing to address a listener's hearing deficiencies or profile, for example, through loudspeaker output. Additional embodiments disclosed herein related to hearing correction systems, products, and methods that measure a specific listener's unique hearing deficiencies or profile.

### OBJECTS AND SUMMARY

This Summary is provided to introduce a selection of representative concepts in a simplified form, which representative concepts are further described below in the Detailed Description. This Summary is not intended to identify key features or essential features, nor is it intended to be used to limit the scope of the claims.

Accordingly, it is an object of certain example embodiments to provide a system and method for acquiring a subject's hearing profile and compensating for detected hearing deficiencies (such as, for example, high frequency hearing loss) in a loudspeaker-based audio system. There are many people who may suffer, either knowingly or unwittingly, from hearing loss who would benefit from the hearing compensation system and method of certain example embodiments. For example, improvements in speech intelligibility should be expected when correcting for high frequency hearing loss since the consonants of speech generally reside in the upper midrange of the human hearing passband while sibilance (“S” sounds) occurs higher in frequency (e.g., 5-10kHz). More generally, certain example embodiments address hearing loss that is detectable.

In a first aspect, a hearing correction system is provided that includes one or more digital signal processing (DSP) modules configured to collectively determine corrected audio output from administered hearing test results (e.g., from a user's measured audiogram). The administered hearing test results can comprise a plurality of hearing loudness threshold levels at respective test frequencies which the test subject detects in response to hearing audio stimuli at those test frequencies. The hearing correction system's corrected audio output can be generated from corrected loudness values at selected correction frequency sub-bands (“correction EQ frequency bands”).

A second aspect provides a hearing correction product comprising a non-transitory computer-readable storage medium and program code embodied on the computer-readable storage medium, the program code executable by a processor to determine corrected loudness values, such as at selected correction frequency sub-bands (“correction EQ frequency bands”), from the administered hearing test results. The administered hearing test results can comprise a plurality of hearing loudness threshold levels at respective frequency sub-bands of a bandwidth detected in response to audio stimuli. The corrected audio output can comprise corrected loudness values, subject to multiband compression, in accordance with corrective magnitude shaping, for example.

A third aspect provides a computer-implemented method, comprising determining corrected loudness values, such as at selected correction frequency sub-bands (“correction EQ frequency bands”), from administered hearing test results that can comprise a plurality of hearing loudness threshold levels at respective test frequencies which the test subject detects in response to hearing audio stimuli at those test frequencies.

The hearing correction system's corrected loudness values can be positive or negative (adding or subtracting signal amplitude) at each of the correction EQ frequency bands. That correction magnitude can be expressed as plus or minus decibel (dB) levels for each correction EQ frequency band. Optionally, all corrective boosts (positive gain) are subject to multi-band, multi-stage compression.

A fourth aspect provides a hearing correction loudspeaker system, comprising one or more loudspeakers and one or more digital signal processing (DSP) modules. The one or more loudspeakers can be configured to administer audio stimuli to a subject in a dual-monaural manner to generate hearing test results, the hearing test results can comprise a plurality of hearing loudness threshold levels at respective frequency sub-bands of a bandwidth encompassed by the audio stimuli. The one or more DSP modules can be configured to collectively determine corrected audio output from the hearing test results, and the corrected audio output can comprise corrected loudness values at the respective frequency sub-bands.

Some embodiments disclosed herein relate to a loudspeaker system and method to measure a specific user's hearing and implement compensatory or corrective processing provided to address the user's hearing deficiencies. The user's hearing acuity may be determined by several different techniques, including but not limited to (i) headphone techniques in which frequency-based hearing thresholds (e.g., 100) are determined; (ii) a loudspeaker system 800 set up as intended for use in an acoustic space to emit tonal stimuli, (iii) inducing, acquiring and interpreting otoacoustic emissions, also known (in part) as the human hearing system's natural response to acoustic stimuli. According to an embodiment, once the user's hearing (e.g., audiometry) profile (e.g., 100, 850, 1310, 1410) is determined, for example by any of these techniques, appropriate signal processing parameters are derived and implemented to generate a corrected audio output signal adjustment (e.g., 1000, 1350, 1450) comprising a selected plurality of correction EQ signals each covering respective frequency sub-bands (“correction EQ frequency bands”). The administered hearing test results 100, 1310, 1410 can comprise a plurality of hearing loudness threshold levels at respective test frequencies which the test subject detects in response to hearing audio stimuli at those test frequencies. The hearing correction system's corrected audio output (e.g., 1350, 1450) can be generated from corrected loudness values at selected correction frequency sub-bands (“correction EQ frequency bands”). Alternatively, in an automotive interior, a corrected automotive audio loudspeaker system may be driven by a corrected audio output signal adjusted for playback in an automotive interior space as a selected position (e.g., the driver's seat).

Some embodiments disclosed herein can relate to a hearing correction loudspeaker system, which can include one or more digital signal processing (DSP) modules configured to collectively determine corrected audio output for a particular subject or user. The corrected audio output can be derived at least in part from administered hearing test results, in some embodiments. The administered hearing test results can include a plurality of hearing loudness threshold levels at respective frequency sub-bands of a bandwidth detected in response to audio stimuli. The corrected audio output can include corrected loudness values at the respective frequency sub-bands.

Some embodiment disclosed herein can relate to a hearing correction loudspeaker system, which can include one or more loudspeakers and a hearing correction system comprising signal processing elements configured to implements corrective signal processing to at least partially compensate for hearing deficiencies of a user based on hearing profile information for the user.

The system can be configured to receive input audio. The signal processing elements can be configured to generate corrected audio output signals based on the input audio and the hearing profile information. The corrected audio output signals can be configured to at least partially compensate for hearing deficiencies of the user. The system can be configured to drive the one or more loudspeakers by the corrected audio output signals to produce audio playback that is corrected to at least partially compensate for hearing deficiencies of the user.

The hearing profile information can be based at least on an age and/or gender of the listener. The hearing profile information can be based on otoacoustic emissions. The hearing profile information can be based on an audiogram specific to the user. The hearing profile information can include information corresponding to a plurality of hearing loudness threshold levels at respective test frequencies which the user detected in response to hearing audio stimuli at those test frequencies. The corrected audio output signals can be generated from corrected loudness values at correction frequency sub-bands. The system (e.g., the signal processing elements) can implement dedicated compression modules for each of the respective frequency sub-bands. The hearing profile information can include composite “better ear” information for each of a plurality of hearing loudness threshold levels at respective test frequencies which the user detected in response to hearing audio stimuli at those test frequencies. In some embodiments, the hearing profile information can include right-ear hearing profile information and left-ear hearing profile information. The signal processing elements can be configured to generate right-ear corrected audio output signals based on the input audio and the right-ear hearing profile information. The signal processing elements can be configured to generate left-ear corrected audio output signals based on the input audio and the left-ear hearing profile information. The system can be configured to drive the one or more loudspeakers to use beamforming provide a right audio zone at a right ear of the user to present right-ear-corrected audio that is corrected to at least partially compensate for hearing deficiencies of the right ear of the user, and/or to use beamforming provide a left audio zone at a left ear of the user to present left-ear-corrected audio that is corrected to at least partially compensate for hearing deficiencies of the left ear of the user. The system can be configured to use the one or more loudspeakers to output test tones for conducting an audiogram hearing test.

Some embodiments disclosed herein can relate to an audio system, which can include one or more loudspeakers and one or more hardware processors which can be configured to access hearing profile information, access audio content, determine modified audio signals based at least in part on the audio content and the hearing profile information, and drive the one or more loudspeakers based on the modified audio signals.

The hearing profile information can be based at least on an age and/or gender of the listener. The hearing profile information is based on otoacoustic emissions. The hearing profile information can be based on an audiogram specific to the user. The hearing profile information can include information corresponding to a plurality of hearing loudness threshold levels at respective test frequencies which the user detected in response to hearing audio stimuli at those test frequencies. The corrected audio output signals can be generated from corrected loudness values at correction frequency sub-bands. The signal processing elements can be implement dedicated compression modules for each of the respective frequency sub-bands. The hearing profile information can include composite “better ear” information for each of a plurality of hearing loudness threshold levels at respective test frequencies which the user detected in response to hearing audio stimuli at those test frequencies. In some implementations, the hearing profile information can include right-ear hearing profile information and left-ear hearing profile information. The signal processing elements can be configured to generate right-ear corrected audio output signals based on the input audio and the right-ear hearing profile information. The signal processing elements can be configured to generate left-ear corrected audio output signals based on the input audio and the left-ear hearing profile information. The system can be configured to drive the one or more loudspeakers to use beamforming provide a right audio zone at a right ear of the user to present right-ear-corrected audio that is corrected to at least partially compensate for hearing deficiencies of the right ear of the user, and/or to use beamforming provide a left audio zone at a left ear of the user to present left-ear-corrected audio that is corrected to at least partially compensate for hearing deficiencies of the left ear of the user. The system can be configured to use the one or more loudspeakers to output test tones for conducting an audiogram hearing test.

The above aspects and still further objects, features and advantages of certain aspects and embodiments will become apparent upon consideration of the following detailed description of example embodiments, particularly when taken in conjunction with the accompanying drawings, wherein like reference numerals in the various figures are utilized to designate like components.

### BRIEF DESCRIPTION OF THE DRAWINGS

The drawings referenced herein form a part of the specification and are incorporated herein by reference. Features shown in the drawings are meant as illustrative of one or more embodiments, and not necessarily of all embodiments, unless otherwise explicitly indicated.

FIG. 1 is an hearing loss plot or audiogram for a first test subject, the audiogram generated by a representative commercial audiometer (e.g., the CheckHearing™ Online Audiometer, headphone method), wherein X's (left ear) and O's (right ear) indicate each ear's hearing thresholds.

FIG. 2 is a graph demonstrating relationships between sound pressure level (x-axis) and loudness (y-axis) for a person with healthy hearing and a person with impaired hearing.

FIG. 3 illustrates a graph plotting input sound pressure level (x-axis) and output sound pressure level (y-axis) to demonstrate wide band dynamic compression principles.

FIG. 4 illustrates a graph plotting input sound pressure level (x-axis) and output sound pressure level (y-axis) to demonstrate examples of amplification/compression compensation for mid-high frequency (e.g., 500 Hz, 1 kHz, 2 kHz, and 4 kHz) hearing loss.

FIG. 5A is a signal flow diagram illustrating an example embodiment of a hearing correction system for carrying out digital signal processing (DSP) with a crossover block dividing the input signal into a selected number of sub-bands or correction EQ frequency bands, and in the illustrated example, three correction EQ frequency bands (low, medium, and high), define a distinct signal path with a dedicated band-dependent Dynamic Range Compression (“DRC”) module.

FIG. 5B is a signal flow diagram illustrating an alternative embodiment of a hearing correction system for carrying out digital signal processing (DSP) with three correction EQ frequency bands (low, medium, and high), where each correction EQ frequency band signal path has a dedicated band-dependent parametric Dynamic Range Compression (“DRC”) and Limiter “lim” modules and a post processing gain block.

FIG. 6A is a process flow diagram illustrating the method steps for an example embodiment and FIG. 6B is a graph illustrating hearing loss with and without correction for a test subject.

FIG. 7 is a diagram illustrating a standard (e.g., Dolby® ATMOS™) loudspeaker setup in a room that may be useful for various embodiments disclosed herein.

FIG. 8 illustrates a dual-monaural audiogram generated from tonal stimuli presented simultaneously via both Polk® R200™ loudspeakers of a left and right stereo pair (e.g. in positions 802, 804).

FIG. 9 is a graph illustrating a dual-monaural audiogram, plotting measured hearing loss (-) which reflects the measurements of FIG. 8 and the expected/corrected hearing loss ( . . . ), the latter with non-normalized hearing correction imposed (with the corrections from col. 6 of Table 2 applied), wherein the dual monaural audiogram was acquired in a physical listening space using Polk® R200™ loudspeakers to emit tonal test stimuli.

FIG. 10 is a graph illustrating hearing correction magnitude for a test subject based on dual monaural audiogram using Polk® R200™ speakers, with plots for raw correction in both ears (-), limited non-normalized correction based on both ears (-), and limited normalized correction based on both ears ( . . . ) with the corrections from Table 2 applied.

FIG. 11 is a screen shot illustrating a DSP control panel with parametric equalizer (EQ) settings for addressing a test subject's dual monaural loudspeaker-based audiogram.

FIG. 12 is a graph illustrating a magnitude response associated with the parametric EQ settings shown in FIG. 11.

FIGS. 13A-13C relate to a system and method for corrective processing for a unique test subject (#2) using an all-in-one stereo (2.0) speaker according to an example embodiment.

FIGS. 14A-14C relate to a system and method for corrective processing for another unique test subject (#14) using the all-in-one stereo (2.0) speaker according to an example embodiment.

FIGS. 15A-15B relate to a system and method for corrective processing for a unique test subject (#14) using a stereo pair of loudspeakers according to an example embodiment.

FIGS. 16A-16C relate to a system and method for corrective processing for a unique test subject (#14) using the stereo pair of loudspeakers according to an example embodiment.

FIGS. 17A-17B relate to a system and method for corrective processing for another unique test subject (#2) using a stereo pair of loudspeakers according to an example embodiment.

FIGS. 18A-18C relate to a system and method for corrective processing for a unique test subject (#2) using the stereo pair of loudspeakers according to an example embodiment.

FIGS. 19A-19C illustrates the DSP settings for the system and method for corrective processing for a unique test subject (#2) using a stereo pair of loudspeakers according to an example embodiment.

FIG. 20 is a signal flow diagram illustrating the sequence of digital signal processing (DSP) steps used in an all-in-one stereo (2.0) speaker system.

FIG. 21 illustrates Age-Related Hearing Loss (Presbycusis) for Men aged 40-80 years old, relative to an 18 year old man.

FIG. 22 illustrates Age-Related Hearing Loss (Presbycusis) for Women aged 40-80 years old, relative to an 18 year old woman.

FIG. 23 illustrates Hearing correction for age-related hearing loss (normalized and limited to 20 dB), for men aged 40-80 years old.

FIG. 24 illustrates Hearing correction for age-related hearing loss (normalized and limited to 20 dB), for women aged 40-80 years old.

FIG. 25 is a signal flow diagram illustrating an alternative embodiment implementing the signal processing to generate a corrected output signal in response to an (a) audio program material input signal and (b) hearing correction parameters, whereby the input audio program signal is divided into a selected number (e.g., N+2) sub bands where n corresponds to the number of test frequencies in an audiogram.

### DETAILED DESCRIPTION

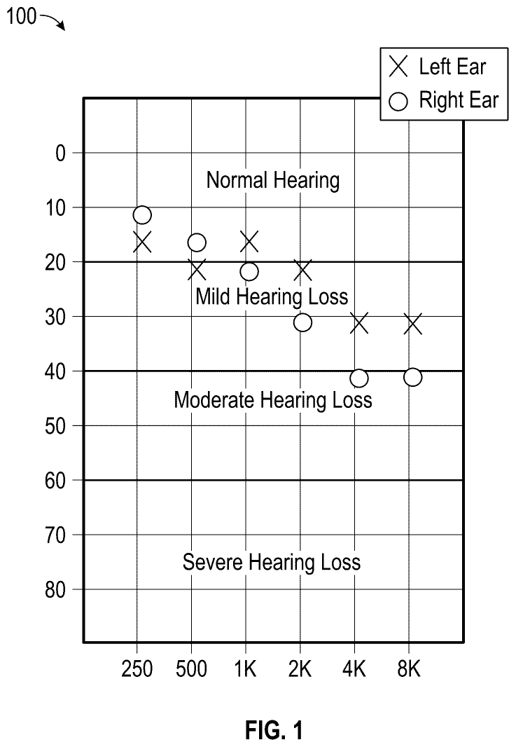

It will be readily understood that the components and features of the example embodiments, as generally described herein and illustrated in the attached FIGS. 1-25, may be arranged and designed in a variety of different configurations. Thus, the following detailed description of certain embodiments of the methods, devices, assemblies, apparatus, systems, products, modules, submodules, etc. of the example embodiments, as presented in the Figures, is not intended to limit the scope of the embodiments, but is merely representative of selected embodiments.

The illustrated embodiments will be best understood by reference to the drawings, wherein like parts are designated by like numerals throughout. The following description is intended only by way of example, and illustrates certain selected embodiments of methods, devices, assemblies, apparatus, systems, products, modules, submodules, etc.

Reference throughout this specification to “a select embodiment,” “one embodiment,” “an example embodiment,” “example embodiments,” “an embodiment,” “embodiments,” or the like means that a particular feature, structure, or characteristic described in connection with the embodiment(s) is included in at least one embodiment. Thus, appearances of the phrases “in a select embodiment,” “in one embodiment,” “in an example embodiment,” “in example embodiments,” “in an embodiment,” “in embodiments,” or the like in various places throughout this specification are not necessarily referring to the same embodiment(s) or only a single embodiment. The embodiments may be, for example, combined with one another in various combinations and modified to include features of one another.

An example embodiment will now be described with reference to a first test subject. The hearing of the first test subject is tested, such as using CheckHearing.org's online audiometer which uses third octave band warble tones (tonal beeps) as stimuli, although it should be understood that other audiometers may be used instead. In the illustrated embodiment of FIG. 1, the testing bandwidth is five octaves, bounded by 250 Hz and 8.0 kHz, encompassing six measurement frequencies (i.e., 250 Hz, 500 Hz, 1.0 kHz, 2.0 kHz, 4.0 kHz, and 8.0 kHz). It should be understood that fewer, additional, and/or alternative frequencies may be used. According to an embodiment, the test procedure involves sequentially presenting the test tones, starting at 250 Hz, at very quiet levels which are intended to be below the first test subject's threshold of hearing. Then, the level of the presented test tone is increased, such as progressively increased in controlled increments, until the test subject can reliably detect the tone and that level is recorded when the test subject or test administrator indicates detection, such as by clicking on the ear icon corresponding to the test frequency and level for the left or right ear under test, or by otherwise indicating the test subject's hearing threshold for that particular test tone. Next, the test subject or administrator advances to the next higher tone (e.g., from 1.0 kHz to 2.0 kHz) and repeats the aforementioned test sequence until the test subject's hearing thresholds for the ear under test, normally (but not necessarily) starting with left, have been determined. This testing routine is followed for testing the subject's other ear to generate a plurality of user-specific unique audiometry test results, corresponding to a plurality of Hearing Loss (HL) metrics for the test subject or user for each ear (e.g., as illustrated in FIG. 1).

It should be noted that equally valid results may be achieved by initiating the test stimuli at relatively high levels and progressively reducing the level until the test subject reports that the test stimulus (e.g., tone) is barely audible or no longer audible. Similarly, it should be understood that the order in which the test stimuli are presented, from low frequency to high frequency or high frequency to low frequency or otherwise, should have no bearing on the validity of the acquired audiogram. That said, in some instances the test stimuli are presented in order of ascending frequency (low to high) and amplitude or signal level (very quiet to louder). The first test subject's audiogram generated in the manner described above is illustrated in FIG. 1. The audiogram exhibits decreasing sensitivity to higher frequencies (especially above 4 kHz). In FIG. 1, “X”s represent hearing thresholds for the left ear and “O”s represent hearing threshold for the right ear.

There are other methods to measure a subject or user's hearing and generate an audiogram (e.g., similar to FIG. 1). Otoacoustic emission-based methods can be implemented using methods such as those described in one or more of U.S. Pat. Nos. 9,497,530, 10,327,064 and 10,327,086, which are incorporated herein by reference for all that they disclose. Thus, otoacoustic methods are suitable for generating the user's or unique listener's Hearing Loss (HL) metrics to be used in the methods and systems disclosed herein for generating correction computations as described below and tabulated in Tables 1 and 2 and then generate the hearing correction filters as part of the algorithms disclosed herein.

While it may be tempting to simply invert an audiogram, such as the audiogram of FIG. 1, to derive a hearing correction filter for an individual's hearing loss, there are several factors that were discovered in applicant's development work to significantly affect binaural hearing which complicate the development of an effective, comprehensive algorithm. First, the extent to which the individual's two ears are matched, or symmetrical, with respect to hearing sensitivity was found to be consequential. Most people do exhibit minor or significant asymmetrical hearing loss. For the first test subject whose audiogram is illustrated in FIG. 1, hearing loss in the right ear (represented by the “O” data points) is significantly more profound than the left ear (represented by the “X” data points) by approximately 10 dB at frequencies of 2 kHz, 4 kHz, and 8 kHz. While hearing aids may be tuned to each individual ear and thereby effectively address asymmetrical hearing loss, stereo or single monophonic loudspeakers cannot do so because interaural crosstalk (IAC) will be quite high. IAC is a measure of how much sound each ear “hears” from an opposing (contralateral) left/right sound source (for the stereo speaker case) or from a single source. Further, when both ears are exposed to a sound source, an individual's experience of overall hearing loss tends to nearly match that of the “better” ear (i.e., the ear having less hearing loss). So applicant's development work has shown that in certain example embodiments, hearing correction intended for speakers (e.g., having relatively high IAC) can primarily address the individual's better hearing ear, as opposed to an average of both ears or the “weaker” of the two ears. This effect was modelled to generate a composite, monaural audiogram that was derived from audiogram data for each test subject's better ear (having lower hearing loss) at each test frequency.

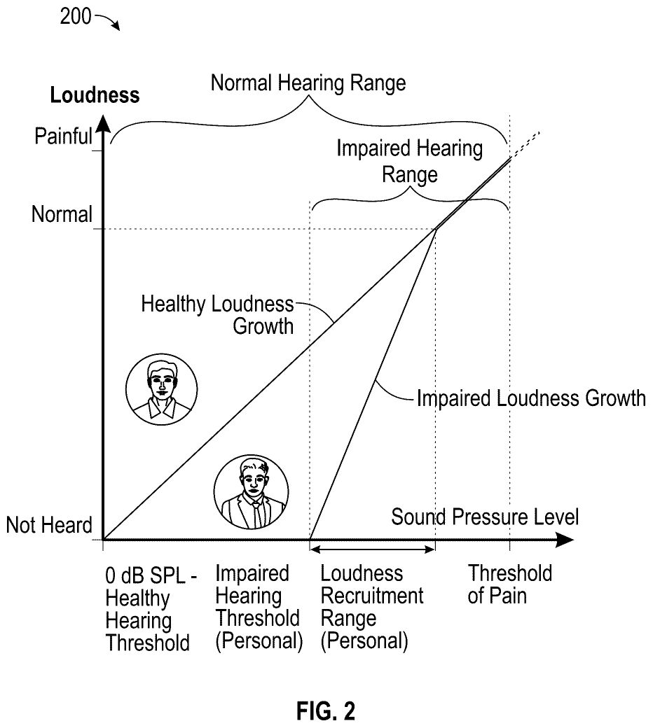

Another consideration for hearing correction involves the dynamics, or dependency on sound level, of hearing perception and its implications on hearing correction. While an audiogram shows an individual's frequency-dependent threshold of hearing, that is the lowest sound level perceived at each test frequency, but hearing perception generally changes with both sound level and frequency. Furthermore, by virtue of an effect called “loudness recruitment”, the perceived loudness of sounds above the threshold of hearing grows faster for people with hearing loss than for those with normal hearing, as illustrated in diagram 200 of FIG. 2. FIGS. 2-4 described below, are reproduced from a technical Whitepaper entitled “Digital Signal Processing for Over The Counter Hearing Aids,” by Alexander Goldin, PHD, Revision 1.2 (March 2023). Graph 200 or FIG. 2 plots sound pressure level (SPL) on the x-axis and perceived loudness on the y-axis. SPL and loudness have a generally linear relationship for a person having healthy ears, at least over the normal hearing range (i.e., 20 Hz to 20 kHz). In the case of the individual with impaired hearing of FIG. 2, external acoustic stimuli are inaudible until the SPL reaches audibility for that individual's impaired hearing, at which level the perceived loudness of sounds linearly increases at a faster rate until eventually matching the perceived loudness of the person with healthy ears at a “normal” loudness level. At SPLs greater than the normal loudness level, the impaired loudness growth and the healthy loudness growth largely overlap one another.

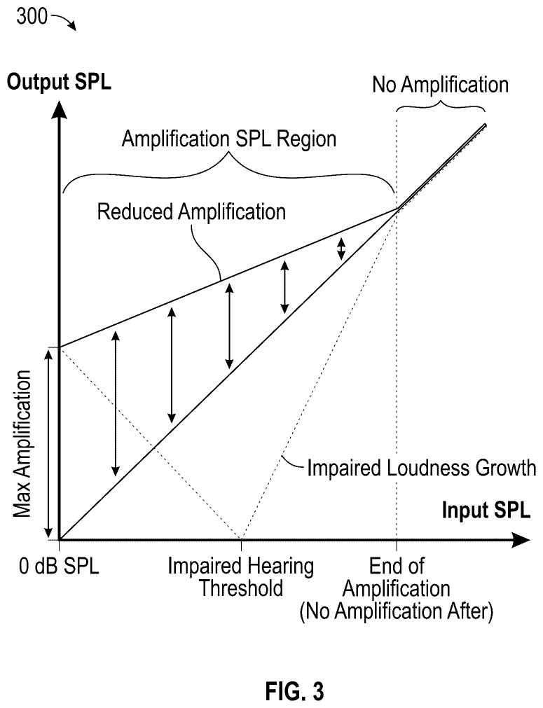

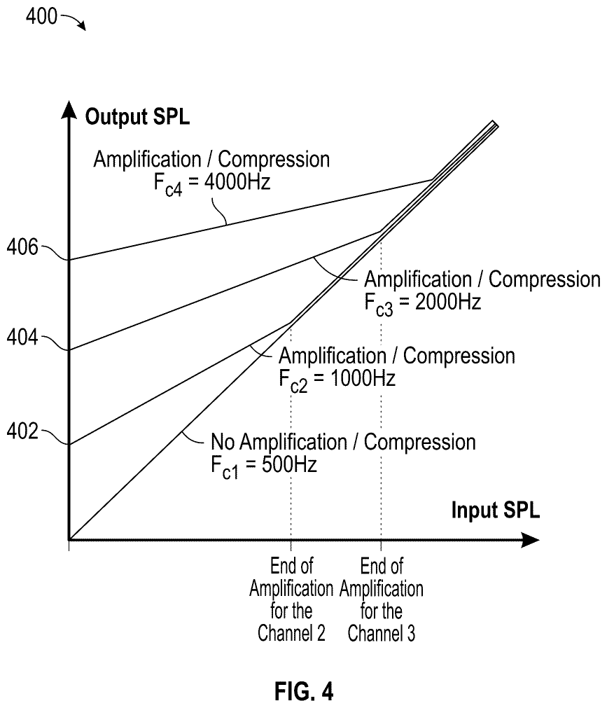

Accordingly, hearing aids can apply progressively lower rates of amplification (gain) with increasing SPL due to loudness recruitment as indicated in plot 300 of FIG. 3. As shown, hearing sensitivity tends towards linear above a certain frequency dependent “input SPL”. A family of such gain vs input SPL for each of the frequency bands associated with the example 4-band hearing tests for an individual suffering from high frequency hearing loss is shown in plot 400 of FIG. 4. While no amplification or compression is needed in the 500 Hz range due to normal hearing in that frequency range, progressively more gain/compression is applied at 1 KHz (1000 Hz) and the succeeding bands of 1 kHz and 2 kHz up to 4 kHz (4000 Hz).

Hearing aids can operate on the basis of partially overlapping frequency “channels”, in some cases numbering from 4-64, each with independent gain and compression parameters that are set in accordance with the individual's hearing loss. For the example shown in plot 400 of FIG. 4, there are four channels, centered over the three octave intervals from 500 Hz to 4 kHz, where a first channel, within the frequency range bounded by approximately 350 Hz and 700 Hz (500 Hz octave band) no amplification or compression is needed while a second channel, for the 1.0 kHz octave band (700 Hz-1.4 kHz), does apply moderate amplification/compression (402).

In some embodiments, hearing correction over loudspeakers can be accomplished in a manner somewhat similar to the hearing aid design discussed herein, as illustrated in plot 400 of FIG. 4. In the hearing compensation algorithm of some embodiments as described and illustrated herein, a user's program material (e.g. streamed music, movie soundtrack, podcast, etc.) to which a hearing compensation algorithm is applied is divided into a selected quantity of (e.g., 3 to 60) frequency sub bands (“correction EQ Frequency Bands”), each of which is addressed independently with respect to dynamic range compensation (DRC) and gain/amplification.

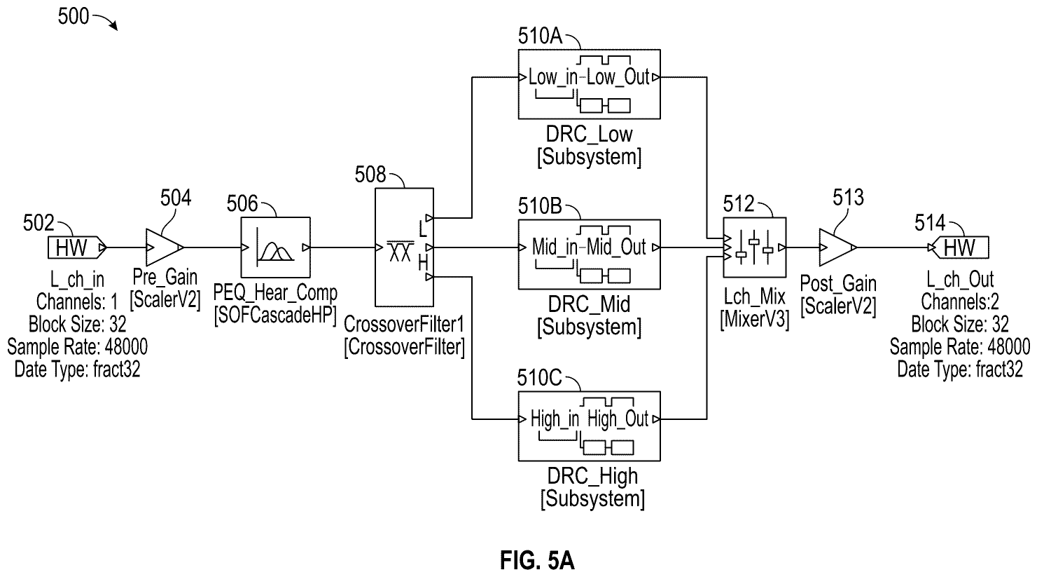

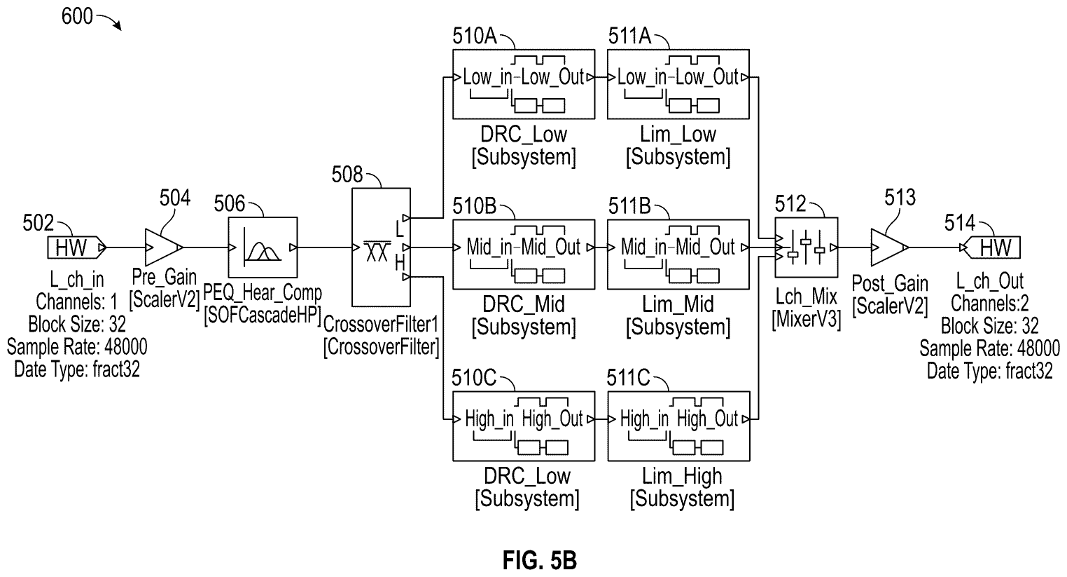

FIGS. 5A and 5B are signal flow diagrams illustrating a representative digital signal processing (DSP) system, generally designated by reference numerals 500 and 600, for carrying out a simplified signal flow for a loudspeaker system, such as for (a) one channel of a stereo pair in a stereo loudspeaker system (e.g., an all-in-one stereo loudspeaker system) or (b) a pair of separate, active loudspeakers (e.g., having a left channel and a right channel). While only a single compression stage is shown, it should be noted that multiple compression stages can be used, for example an initial stage characterized by a lower compression rate (such as 2.0:1) and a relatively low threshold (e.g. −30 dBFS or lower) followed by a limiter (e.g., a higher compression such as of about ˜10:1 and a higher compression threshold, such as of approximately −1.5 dBFS), which can provide improved audio performance over single stage compression. FIG. 5A can also apply to a monophonic (e.g., single-channel) loudspeaker (e.g., an active loudspeaker). As noted elsewhere, the single-stage compression signal flow illustrated in FIG. 5A can be replaced with or augmented with a multistage compression signal flow.

The DSP system 500 shown in FIG. 5A provides for signal flow representing the signal processing applied to program material for hearing compensation/correction purposes according to an example embodiment. The associated digital signal processing (DSP) settings of the DSP system 500 can compensate for or correct the individual user's hearing profile. FIG. 5A illustrates an example embodiment of a sequence in which the signal flow's constituent processes are implemented. First, just downstream of input module 502 (e.g., HW, L_ch_in, Channels: 1, Block Size: 32, Sample Rate: 48,000, Data Type: fract 32) is a gain module 504 (e.g., L_ch_gain, ScalerNV2) which can compensate for any net gain associated with downstream processing, in particular the next Parametric EQ module 506 (e.g., PEQ_hear_comp, SOFCascadeHP). Parametric EQ module 506 can generate a full-bandwidth magnitude domain filter that can be based on the inverse of a selected individual's hearing profile or audiogram (e.g., an inverted version of audiogram 100 for a test subject or user). The output of Parametric EQ module 506 can be input to a “crossover” filter module 508 which can subdivide the audio passband of that signal into multiple sub-bands (e.g., so crossover 508 as illustrated in the example embodiments of FIGS. 5A and 5B can define the selected number (e.g., 3) of “correction EQ frequency bands”). The three signals from crossover 508 are then provided as inputs to independent dynamic range compression (DRC) modules 510A (e.g., DRC_low, subsystem), 510B (e.g., DRC_mid, subsystem), and 510C (e.g., DRC_high, subsystem). Any suitable number of frequency bands and corresponding DRC modules 510 can be used, such as 2, 3, 4, 5, 6, 8, 10, 12, 15, 20, 30, 40, 60, 80, 100, or more.

Each parametric DRC module 510A, 510B, and 510C can operate on a single dedicated frequency band—in this example low, medium, and high, respectively—to effectively limit the applied gain when input levels in that specific frequency band are sufficiently high and in some cases to function as a pass-through (applying no gain or attenuation) such as when input levels are relatively low. Loudness recruitment (e.g., as illustrated in FIG. 2), which generally is frequency dependent as explained previously, can be addressed by this set of dynamic range compressor (DRC) modules 510A, 510B, and 510C. Following the band-dependent DRC modules 510A, 510B, and 510C is a mixer module 512 for reconstituting the signal, followed by gain module 513, and an output module 514 (e.g., L_ch_out, Channels: 2, Block Size: 32, Sample Rate: 48,000, Data Type: fract32).

It should be noted that the embodiment of FIG. 5A presents the signal flow for the left channel of a two-channel configuration and that the right channel's signal flow (including its associated DSP settings) can be identical or similar unless interaural crosstalk (IAC) is sufficiently low to permit binaural hearing compensation, and hence address asymmetric hearing loss (AHL), which is a common condition that comprises significant disparities in the hearing ability between an individual's two ears. Among the ways in which sufficiently low IAC may be achieved is to use InterAural Crosstalk (“IAC”) cancellation, which can use a pair of Stereo Dimensional Array (SDA™) loudspeakers, which provide substantial InterAural Crosstalk (“IAC”) cancellation at a listening position, as described and illustrated in U.S. Pat. Nos. 10,327,064 and 10,327,086, which are incorporated herein by reference for all that they disclose.

There are alternative ways of achieving high IAC cancellation and/or low IAC (e.g., for binaural hearing correction with stereo speakers). For example, one way to provide sufficient IAC cancelation for binaural hearing correction with stereo speakers is to utilize electronic SDA (eSDA), a method of processing two channel signals which includes the derivation of SDA effect signals and cross-mixing them to the opposing channel (e.g. left SDA effect mixed with right “main” signal, and/or right SDA effect mixed with left “main signal). U.S. Patent Application Publication No. 2023/0319478 describes methods for implementing eSDA and is incorporated in its entirety herein by reference for all that it discloses. Audio systems, such as stereo (or home theater) systems incorporating an audio-video receiver (AVR), can provide binaural hearing compensation by including hearing compensation processing in conjunction with eSDA, such as in the AVR's audio signal processing. Establishing the listener's location, by any of various means, relative to the stereo loudspeakers permits binaural audio delivery in conjunction with eSDA or SDA loudspeakers characterized by high IACC, permits AHL compensation, such as for off-axis locations. U.S. patent application Ser. No. 17/933,661 (which is incorporated by reference herein in its entirety) and U.S. patent application Ser. No. 18/903,481 (which is incorporated by reference herein in its entirety) disclose details relating to establishing a listeners location. For example, in some embodiments the system can include a camera or other sensors that can be used to determine one or more listener locations. Video or image analysis can be used to determine one or more listener locations, in some implementations.

Another manner of achieving sufficiently low IAC for addressing asymmetrical hearing loss (AHL) includes multi-element beam steering line arrays, for example in which the beam steering array system is capable of locating an individual's ears, e.g., via camera based head-tracking, or user input, or wearable sensors or markers, or other means, and identifying them as micro-zones and, accordingly, delivering two-channel audio to the individual's ears for binaural hearing correction. Multi-element beam-steering loudspeaker arrays can permit binaural hearing correction over loudspeakers. U.S. patent application Ser. No. 18/903,481 (which is incorporated by reference herein in its entirety) discloses details relating to beamforming features that can be used to send different audio to different locations using loudspeakers. The beamforming and beam steering features can produce areas of constructive and/or destructive interference so that different audio is presented at a first area (e.g., at a first micro-zone at a listener's left ear) than the audio that is presented at a second area (e.g., at a second micro-zone at a listener's right ear). Accordingly, the system can have or access hearing profile information (e.g., an audiogram) for the right and left ears of a listener. The system can then determine corrected audio for the right ear (e.g., based on the left-ear audiogram or hearing profile) and different corrected audio for the left ear (e.g., based on the left-ear audiogram or hearing profile). The system can then present the audio that is right-ear-corrected to the right ear of the user (e.g., using beamforming or beam steering), and the system can present the audio that is left-ear-corrected to the left ear of the user (e.g., using beamforming or beam steering).

When operating on program signals intended for reproduction by a stereo all-in-one loudspeaker system, a pair of active speakers or a loudspeaker system driven by an AVR (audio-video receiver) which includes hearing correction processing, the DSP settings for each left (L) and right (R) channel input signal can be identical or similar, in the absence of an audio system capable of providing low IAC, since the interaural crosstalk for such systems can be quite high. That is, when each L/R ear is exposed to relatively high levels of the opposing (contralateral) stereo (R/L) channels, asymmetric hearing loss can be challenging to be effectively addressed by such systems. An example embodiment will show how systems that provide substantial interaural crosstalk cancellation (IACC) may be configured to address asymmetric hearing loss.

The DSP system 500 of FIG. 5A is illustrated as a three-band system (with low/med/high correction EQ frequency bands) each having dedicated dynamic range compression selected for that correction EQ frequency band. Increasing the number of frequency bands to five or more according to further example embodiments can improve the system's flexibility to address a wide range of user otological hearing profiles, particularly ones that feature narrow band anomalies. There could be many more (e.g., sixty or any suitable number) correction EQ Frequency Bands, each having its own DRC settings.

While single-stage compression, shown for example by 510A in FIG. 5A, may be sufficient for achieving a targeted range of band-dependent signal levels through the native signal's dynamic range, there may exist objectionable audio artifacts associated with simple compressors, especially when employed as limiters. In particular, distortion products such as clipping (manifested as audible, high THD, level fluctuation or pumping), may result when input levels greatly exceed a DRC's threshold when the DRC is configured to function as a limiter, characterized by a relatively large compression ratio and a short-duration attack. Multi-stage compression, shown in FIG. 5B, can mitigate these distortion artifacts by more gently reducing excessive band-dependent signal levels in advance of the downstream limiters (e.g., “lim” modules, 511A, 511B and 511C in FIG. 5B). While two-stage compression (as illustrated in FIG. 5B) can provide substantial improvements in audio signal quality compared to single-stage DRC (as illustrated in FIG. 5A), additional compression stages (e.g., two or more upstream of a final limiter stage, e.g., 511B shown in FIG. 5B) can provide further improvements.

Skilled practitioners in the fields of audio processing, human hearing and hearing aids will recognize that additional or alternative processing may provide further benefits when applied to loudspeaker-based hearing compensation systems (a focus of this work). As its name implies, a side-chain compressor is triggered by an audio or control signal other than the one on which it operates. For the present application, side-chain compression (not shown) may provide a more natural sounding result with higher intelligibility than a single or series of DRCs. The control signal for a side-chain compressor in this context could be the overall input signal, upstream of the dividing network (e.g., 508). Use of a parametric expander, which effectively boosts signals whose detected levels are below a prescribed expansion threshold, can further improve clarity and intelligibility beyond the capabilities of a hearing correction PEQ (506) operating in concert with band-dependent DRCs (510). Besides an expansion threshold, parametric expanders can offer control of expansion rate in the form of a ratio (e.g. 2:1, 5:1, 20:1 etc.), and a maximum gain setting. Side-chain expansion further offers even finer signal conditioning for improved audio performance and intelligibility. Finally, a “compander”, as the name implies, offers a combination of both compression and expansion and can be configured for side-chain triggering. They optionally may be incorporated in the loudspeaker-based hearing compensation systems disclosed herein.

Still further regarding the signal flow of the DSP system (e.g., 500 or 600 of FIGS. 5A, 5B) the crossover filter module 508 can be configured such that the three sub-bands into which the full-range input is divided sum to a magnitude response that exactly or substantially matches the input to the crossover filter module 508. In an embodiment of the system and method, the high and low pass filters (frequency, Q, order) comprising the crossover filter module 508 are configured to ensure that the crossover filter module's output signals sum to that magnitude response (e.g., exactly or substantially matching the input to crossover filter module 508). This crossover output summing match characteristic can apply to any such crossover filter or dividing module of an arbitrary number of (e.g., 3-60) output channels or correction frequency sub-bands.

The DSP systems (e.g., 500 or 600 of FIGS. 5A, 5B) and other embodiments described herein can be operatively connected to and communicate with a processor or controller and an associated memory. The memory may store, for example and without limitation, programming instructions and data used by the processor during program execution, including parameters and values, and other information. In one or more embodiments, the processor and the memory are local, such as local storage. In another embodiment, the processor and/or the memory can be a remote processing unit and/or database (e.g., loaded on a smartphone or a computer or a tablet) connected to the DSP system using wired or wireless connections.

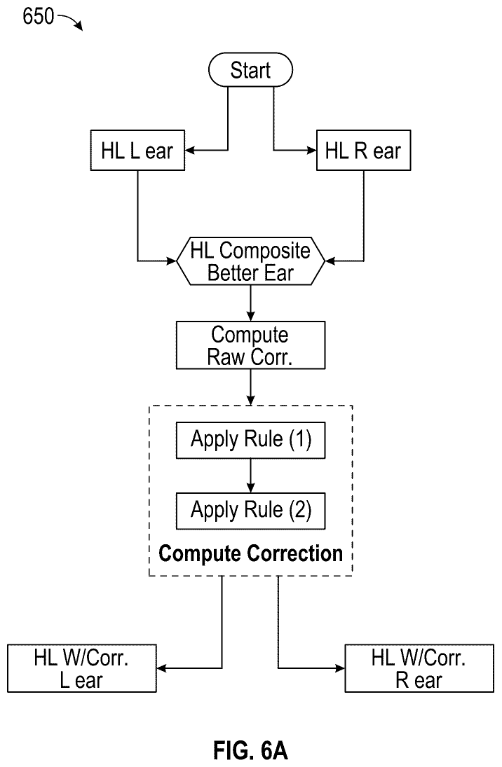

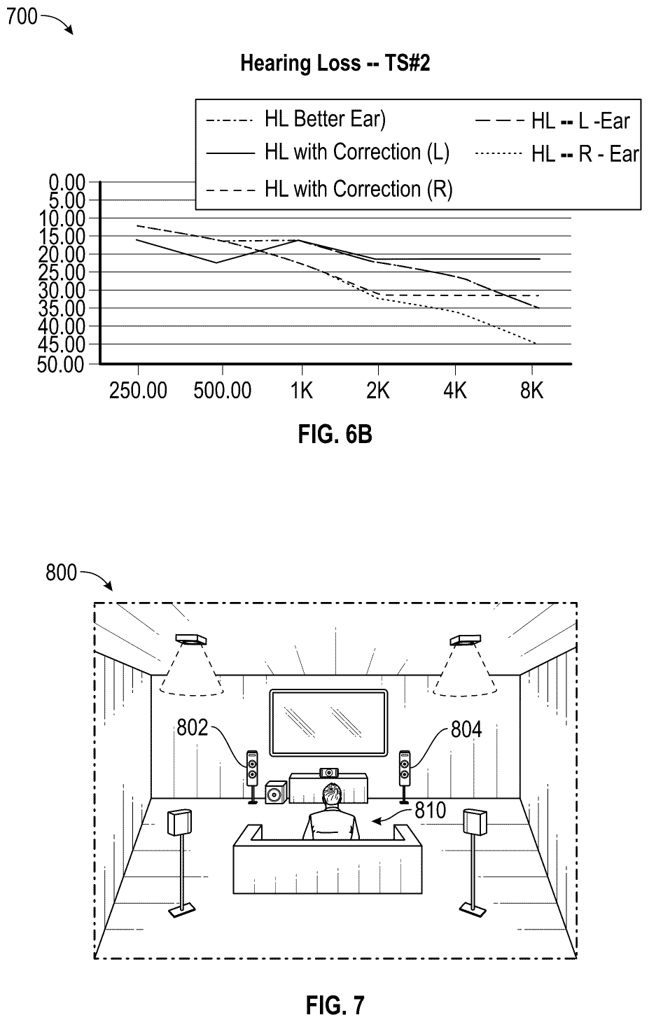

In accordance with the method, a representative binaural hearing profile (or audiogram) may be acquired using headphones, and that hearing profile can be processed to generate a corrected audio output signal adjustment. FIG. 1 illustrates a first test subject's audiogram (or hearing profile), which clearly displays asymmetric hearing loss (“HL”) with progressing frequency, or simply high frequency hearing loss. After the first test subject's audiogram is acquired (see FIG. 6A), any appropriate offsets for compensating for the headphone's frequency response may be applied (not shown). Next, a comparison of the two ears' hearing losses at each test frequency can be made, and the lower of the two values can be adopted to represent the individual's hearing loss at that frequency. With reference to FIG. 1, for the first test subject, at 250 Hz the right ear's hearing loss (HL) of about 12 dB is lower than (i.e., not as bad as) the left ear's hearing loss (HL) of about 16 dB. The HL value at 250 Hz is determined to be the lower of the two values (i.e., 12 dB<16 dB). At 500 Hz, the right ear has the lower hearing loss value, which can be used instead of the higher value of the left ear. At frequencies of 1 kHz and above, the left ear's HL values are substantially lower and consequently the HL of the first test subject can be based on the left ear as the better ear over that passband.

Table 1 shows the hearing profile for the first test subject and the results of computations in accordance with a hearing compensation algorithm according to an example embodiment. Correction in Table 1 is based on a pure tone (PT) threshold test administered over headphones.

TABLE 1

| | | | correction based on better hearing ear (L or R) | | | | |
|---|---|---|---|---|---|---|---|
| | | | HL | | correction | HL with | HL with |
| | | | (composite, | Raw | based on | correction | correction |
| freq (Hz) | HL, L ear | HL, R ear | better ear) | Correction | better ear | (L) | (R) |
| 250.00 | 16.00 | 12.00 | 12.00 | −9.17 | 0.00 | 16.00 | 12.00 |
| 500.00 | 22.00 | 16.00 | 16.00 | −5.17 | 0.00 | 22.00 | 16.00 |
| 1k | 16.00 | 22.00 | 16.00 | −5.17 | 0.00 | 16.00 | 22.00 |
| 2k | 22.00 | 32.00 | 22.00 | 0.83 | 0.83 | 21.17 | 31.17 |
| 4k | 26.00 | 36.00 | 26.00 | 4.83 | 4.83 | 21.17 | 31.17 |
| 8k | 35.00 | 45.00 | 35.00 | 13.83 | 13.83 | 21.17 | 31.17 |
| HL (PT6) | 22.83 | 27.17 | 21.17 | | | | |
| HL avg | 25.00 | | | | | | |

First, (see FIG. 6A) the hearing loss (HL expressed as “dBHL”) for each ear and at each of the six test frequencies can be tabulated with reference to the acquired audiogram (FIG. 1). The data have been adjusted to reflect the test headphone's acoustic magnitude (frequency) response for a more accurate representation of the individual's hearing loss. For each ear, the average HL over a selected number (e.g., six or any suitable number) of test frequencies can be computed. Optionally, the audiogram test signals comprise six “pure tone” test signals or six warble tones varying or warbling (within a narrow frequency range) about each center frequency. In the method, the test subject or user's hearing loss (HL) isn't the same in both ears, and HL for the better ear (i.e., the ear having lesser hearing loss) can be compared with the average HL when using a pure tone, six frequency test (“PT6” is computed as the average magnitude of the hearing losses for the test subject's better ear over the six frequencies).

In the example of Table 1, The first test subject's right ear “PT6” hearing loss is substantially greater than the left ear's (27.17 dBHL vs 22.83 dBHL). The average hearing loss for both ears (combined) is computed to be 25.00 dBHL.

According to an example embodiment of the method, the hearing loss (HL) of the better ear (i.e., the ear having lesser hearing loss) is compared with the average HL when using a pure tone, six frequency test (“PT6”), which is the average of the hearing losses of the better ear(s) over the six frequencies. In Table 1, Hearing Loss HL (PT6) of the “composite better ear” is computed by summing the lower HL value at each frequency irrespective of whether the lower HL value is for the left ear or the right ear. Thus, the composite better ear computation may sum HL values from both the left ear and the right ear on a frequency-by-frequency basis. For example, in Table, 1, the composite better ear value is calculated as (12.00[HL, R ear]+16.00[HL, R ear]+16.00 [HL, L ear]+22.00[HL, L ear]+26.00[HL, L ear]+35.00[HL L ear])/6, which equals 21.17.

More specifically, as seen in the 4^{th} column (HL (composite, better ear)) of Table 1, the hearing loss composite value for the better ear is tabulated for each of the six frequencies identified in this example embodiment. Then, “raw correction” values may be computed on this basis, by comparing the “better ear's HL to the composite HL PT6 value of 21.17 dB: at 250 Hz the raw correction for the test subject is −9.17(12.00[HL, R ear]−21.17), while at 500 Hz it is −5.17(16.00[HL, R ear]−21.17). The raw correction hearing loss for the better ear at 1 kHz is −5.17(16.00[HL, L ear]−21.17), and at 2 kHz it is 0.83(22.00[HL, L ear]−21.17), while at 4 kHz it is 4.83(26.00[HL, L ear]−21.17), and at 8 kHz it is 13.83(35.00[HL, L ear]−21.17).

Accordingly, a “raw correction” is then computed (as shown in the 5^{th} column of Table 1). The raw correction equals the HL PT6 composite average subtracted from the better ear's HL for each test frequency. In the illustrated example, the raw correction at 1.0 kHz is 21.17 dBHL subtracted from 16.0 dBHL dB, equal to negative 5.17 dB (−5.17 dB).

In an example embodiment, the appropriate applied or “net” correction is derived from the raw correction by applying one or more of the following parameters (or rules): (1) when the raw correction is less than zero, no correction shall be applied or implemented and (2) if the raw correction exceeds a prescribed limit, such as 20 dB or another prescribed limit, the correction shall be set to that limit, e.g., 20 dB.

A second example rule or parameter (2) ensures that the host product's active (signal processing+amplification) system operates well within its dynamic range constraints and that any risks of distortion are substantially mitigated. Furthermore, experience based on conducting a hearing study on a range of test subjects suggests that 12-20 dB of corrective gain is sufficient, even for test subjects whose computed raw correction value may greatly exceed 20 dB.

In some embodiments, a first rule or parameter (1) (not shown in FIG. 6A) ensures that no attenuation (i.e., negative correction) is applied over those test frequencies when the subject's overall average hearing loss exceeds that within a particular frequency band. Instead, while it would be possible to normalize the correction curve in accordance with the subject's lowest hearing loss (e.g., at 250 Hz, HL=12 dB for the first test subject, yielding a raw correction of −9.17 dB), doing so would mean implementing broadband gain of 9.17 dB in this instance, which would effectively make the post processed program signal much higher in level compared to the native (pre-processed) program signal. While there is a gain offset control (“makeup gain”) in the signal flow upstream of where the correction filters are applied (or downstream, or both upstream and downstream) which could compensate for this gain, doing so would effectively push the implemented correction above the prescribed limit (e.g., 12-20 dB) in some circumstances and may thereby excessively constrain the correction magnitude when the second rule or parameter (2) is enabled. That said, this alternative approach to managing gain and correction is reasonable in some cases and has been found to be effective for many subjects. As such, it is within the scope of this disclosure to include this and other alternatives to the computational process and parameters (or rules) detailed presently.

In the illustrated embodiment, no compression (or expansion) is applied for any frequency bands that are attenuated by simply setting the correction to 0 dB when the nominal “Raw” correction is negative. Normalizing the corrective gain, in accordance with a test subject's lowest hearing loss associated with their composite audiogram, has been found to be an effective approach. However, it is reasonable to consider alternatives to this approach and the scope of the present disclosure does not preclude these and other permutations of such implementations.

In Table 1, the column entitled “correction based on better ear” shows the results of the implementation of parameters (1) and (2) for the first test subject, a corrective gain of 0.83 dB, 4.83 dB and 13.83 dB applied at 2 kHz, 4 kHz and 8 kHz, respectively. The final two columns show the expected hearing loss for each ear when the correction is applied. The data in Table 1 for HL-L-ear, HL-R-ear, HL with correction based on better ear, HL with correction (L), and HL with correction (R) are plotted in FIG. 6B.

### Additional Embodiment With use of Loudspeakers to Emit Test Stimuli

In some cases, test stimuli are delivered (presented) via headphones, thereby precluding or impeding any acoustic artifacts of the physical space from affecting the results and ensuring near-zero interaural crosstalk. Due to the latter, binaural hearing profiles can be acquired over headphones as opposed to loudspeakers. However, when a hearing profile is acquired in this manner and applied to loudspeakers, corrective filters may fail to properly address loudspeaker performance characteristics or room effects that may affect hearing acuity in-situ. For example, the loudspeakers may exhibit a rising magnitude response characteristic which would mitigate the subject's (listener's) high frequency hearing loss. However, if the hearing correction signal processing parameters are determined using headphones to address a test subject's high frequency hearing loss, the correction curve when coupled with the loudspeaker's inherent response may result in in excessive high frequency gain, depending in part on the room's acoustic properties.

Alternatively and by contrast, an acoustically “dead” room (e.g., excessively damped, characterized by greatly attenuated surface reflections and/or a short reverberation time) in which loudspeakers whose semi-anechoic measured magnitude response is substantially neutral or “flat” are operating can serve to exacerbate the subject's high frequency hearing loss. By using the loudspeakers themselves, set up as intended in-situ relative to the listener's preferred seating location, any major issues associated with the loudspeakers or the acoustic space in which the loudspeakers are operating may be avoided (circumvented) altogether. While use of loudspeakers can preclude acquisition of a binaural hearing profile in some situations, corrective filters for each ear can be identical even if the subject exhibits significant asymmetric hearing loss, in some implementations.

In accordance with example embodiments, there are several techniques for determining the corrective filters when using loudspeakers for presenting test stimuli. Among them is using as a basis the average hearing loss determined by presenting test tones via each left and right speaker separately and recording the hearing threshold for each ear with the ipsilateral (same-side) speaker, of a stereo pair, presenting the stimuli. Alternatively, the correction parameters can reasonably be based on the “better” ear at each test frequency when a binaural audiogram is acquired by playing the test tones on each loudspeaker and determining the hearing threshold in accordance with the ipsilateral method described herein.

Over the course of a hearing study conducted by the inventor, it has become clear that while the previously mentioned methods do have merit, in example embodiments the hearing correction processing parameters optimally can be based on an audiogram that is acquired by presenting the test stimuli over both loudspeakers to both ears simultaneously. This method may be described as “dual-monaural”, as opposed to binaural, since both ears are exposed simultaneously to the tonal stimuli which are presented by multiple loudspeaker channels, again simultaneously. Still another method of another embodiment involves blocking either ear (using an earplug or similar) and generating an audiogram for the other, open ear with the test stimuli presented, for example, by the ipsilateral loudspeaker. This method, while not without merit, can impose unnatural conditions on the individual and tends to result in hearing correction parameters that are substantially similar to the first or second methods outlined above. An experienced practitioner of audio, acoustics and hearing would, when having access to this disclosure, would recognize that these and other permutations of these audiogram acquisition methods are reasonable for purposes of providing hearing correction over loudspeakers. Accordingly, the present disclosure encompasses a multitude of such methods.

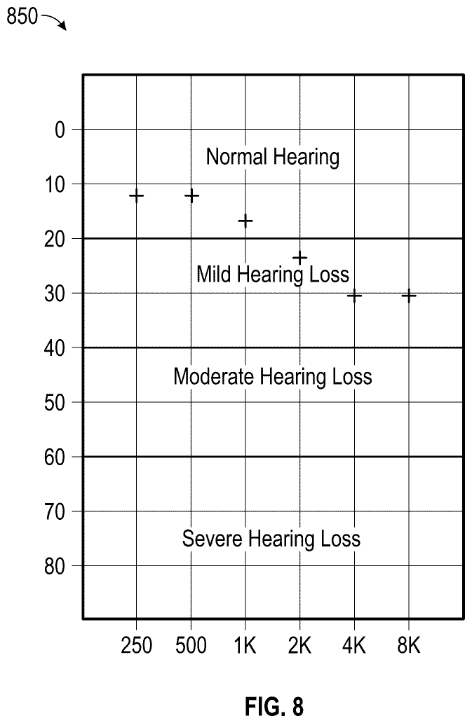

A detailed example method for establishing the hearing correction parameters using loudspeaker setup 800 may generally resemble the standard Dolby recommendations for a loudspeaker system setup as shown in FIG. 7. In the x.y.z setup (typically 5.1.2 for a Dolby Atmos or DTS-x system) illustrated in FIG. 7, only the main left and right loudspeakers (802, 804) present hearing correction test signals for most applications. However, it should be appreciated that the center channel, surround speakers and height channel speakers (or any other suitable speakers) too may alternatively or additionally be used for this purpose. Both of the front stereo speakers (front left 802 and right 804) present the tonal stimuli with the test subject 810 seated in a preferred listening location, such as on the couch facing the front speakers 802, 804 and video monitor (again with reference to the setup of FIG. 7). The test subject 810 indicates when the tonal stimulus, at each frequency, is barely audible, for example, and an audiogram (for example, which generally resembles FIG. 8) is generated. Any other audiogram approach can be used, such as those described herein.

In another embodiment, the hearing test described herein is conducted with use of a specially designed smart-phone application. Alternatives to using a smart-phone application include administering the hearing test using the host loudspeaker system's remote control over a specialized user interface, with use of a video monitor (on-screen display) for guiding the user through the test and showing test results. The aforementioned method may be adapted for use with a soundbar, active speaker and/or an AVR/passive-speaker based systems.

Referring to Table 2 (below) next, data extracted from the acquired dual-monaural audiogram is used to determine the digital signal processing (DSP) settings that address the test subject's in-situ hearing profile. “Raw” correction for each test frequency is computed by comparing hearing loss at each frequency to the dual monaural hearing loss (e.g., “PT6”) average. Then the applied correction is equal to the raw correction (col. 3) unless the raw correction is negative, in which case the applied correction shall be set to be zero. If the raw correction exceeds the prescribed limit (generally 12-20 dB), the applied correction magnitude shall be set to that prescribed limit.

FIG. 8 illustrates a dual-monaural audiogram from speakers in a room as generated from tonal stimuli presented simultaneously via both Polk 50^{th} Anniversary Speaker loudspeakers of a stereo pair (e.g., positioned as are speakers 802, 804 in FIG. 7). From the resulting audiogram (850 in FIG. 8), dual monaural hearing thresholds for the subject or user (e.g., at position 810) are tabulated, and corrective magnitude filters computed. Table 2 shows the results of computing the raw, normalized, limited/normalized, and the limited/non-normalized (applied) correction in columns 3-6 respectively. Table 2 shows results from computations of hearing correction for the first test subject using a loudspeaker system for presenting test stimuli in a physical space.

TABLE 2

| | | | | | | | 8 |
|---|---|---|---|---|---|---|---|
| | | | | 5 | | 7 | HL with |
| | | | | limited, | 6 | HL with | non-norm, |
| | | 3 | 4 | norm. | limited, | lim, norm. | lim. |
| | 2 | raw | norm. | correction | non-norm | correction | correction |
| 1 | HL-both | Correction | correction | based on | correction | both ears | both ears |
| freq (Hz) | ears | (both ears) | (both ears) | both ears | both ears | (L + R) | (L + R) |
| 250 | 10.00 | −9.33 | 0.00 | 0.00 | 0.00 | 10.00 | 10.00 |
| 500 | 10.00 | −9.33 | 0.00 | 0.00 | 0.00 | 10.00 | 10.00 |
| 1k | 16.00 | −3.33 | 6.00 | 6.00 | 0.00 | 10.00 | 16.00 |
| 2k | 20.00 | 0.67 | 10.00 | 10.00 | 0.67 | 10.00 | 19.33 |
| 4k | 30.00 | 10.67 | 20.00 | 15.00 | 10.67 | 15.00 | 19.33 |
| 8k | 30.00 | 10.67 | 20.00 | 15.00 | 10.67 | 15.00 | 19.33 |
| HL (PT6) | 19.33 | | | | | 11.67 | 15.67 |
| HC avg | | 0.00 | 9.33 | 7.67 | 3.67 | | |

With reference to the third column (3) of Table 2, the raw (or gross) correction (f1)=HL(both ears)−HL(PT6) (wherein HL (PT6)=19.33).

As shown in Table 2, the computation shown in the fourth column (4) normalizes the raw correction for both ears (column 3) based upon the test subject's computed raw correction for their lowest hearing loss value in accordance with the dual monaural audiogram (see, e.g., FIG. 8 and column 2). In this example, at 250 Hz and 500 Hz, the hearing loss is lowest (10.00 dBHL) which provides a raw correction value of negative 9.33 dB (col. 3). Thus, the constant negative 9.33 dB subtracted from the raw correction values in Col. 3 yields the normalized correction for both ears shown in Col. 4 (i.e., zero) because of rule or parameter one, as described above. The fifth column (5) provides limited, normalized correction for both ears, which involves application of the rules or parameters (1) and/or (2) to the normalized correction values of the fourth column (4). In the example computation of Table 2, the fifth column (5) provides limited, normalized correction for both ears. In this case, for the maximum gain parameter, a maximum gain, or limit, of 15 dB has been imposed on column (4).

The sixth column (6) provides limited, non-normalized correction for both ears. As such, column (6) reflects the application of both parameters (1) and (2), again with a 15 dB limit imposed. It may be noted that the hearing correction average gain over the six test-frequency stimuli is 7.67 dB and 3.67 dB respectively for the normalized (column 5) and non-normalized (column 6) hearing correction. Finally, columns 7 and 8 show the expected HL when normalized and non-normalized limited hearing correction is applied. Not surprisingly, given the substantially greater hearing correction average gain for the former, the expected hearing loss associated with limited, normalized correction of 11.67 dBHL is substantially lower than that of the limited, non-normalized hearing correction (15.67 dBHL).

In an example embodiment, a magnitude compensation factor based on the correction magnitude averaged over the six test frequencies is applied. As indicated in Table 2, in accordance with this embodiment 3.67 dB of attenuation is applied in order to compensate for the average correction magnitude of 3.67 dB associated with the limited, non-normalized correction for both ears (column 6). It should be noted that in accordance with FIG. 5A [504, 513] that any attenuation may be applied upstream or downstream of

magnitude shaping (EQ) or the DRC/Lim modules when the normalized, limited correction is implemented. While a nominal compensatory attenuation value may be computed as presented here, attenuation values substantially lower (down to 0 dB) or greater may also be applied for providing effective hearing correction over loudspeakers for some test subjects. Furthermore, it should be apparent to someone experienced in the audio, acoustics and hearing/audiology fields that compensatory attenuation and/or makeup gain may be applied upstream (before) or downstream (after) magnitude shaping (EQ) or DRC/limiting, so long as effective hearing correction for an individual is achieved at a playback level comparable to the native, unprocessed signal.

While it may be reasonable to apply “normalized” correction, which in the example of Table 2 includes a leveling compensation factor of 9.33 dB to match the smallest magnitude hearing loss over the PT6 range whose results of this calculation appear in column 3 of Table 2, in applicant's development work, another embodiment has shown favorable results when negative HL values are set to zero, in accordance with the computational algorithm detailed herein for the applied correction (column 6 of Table 2). However, some test subjects may benefit from using a correction based on normalization and the present disclosure encompasses both of these and other possible permutations of a derived hearing correction algorithm.

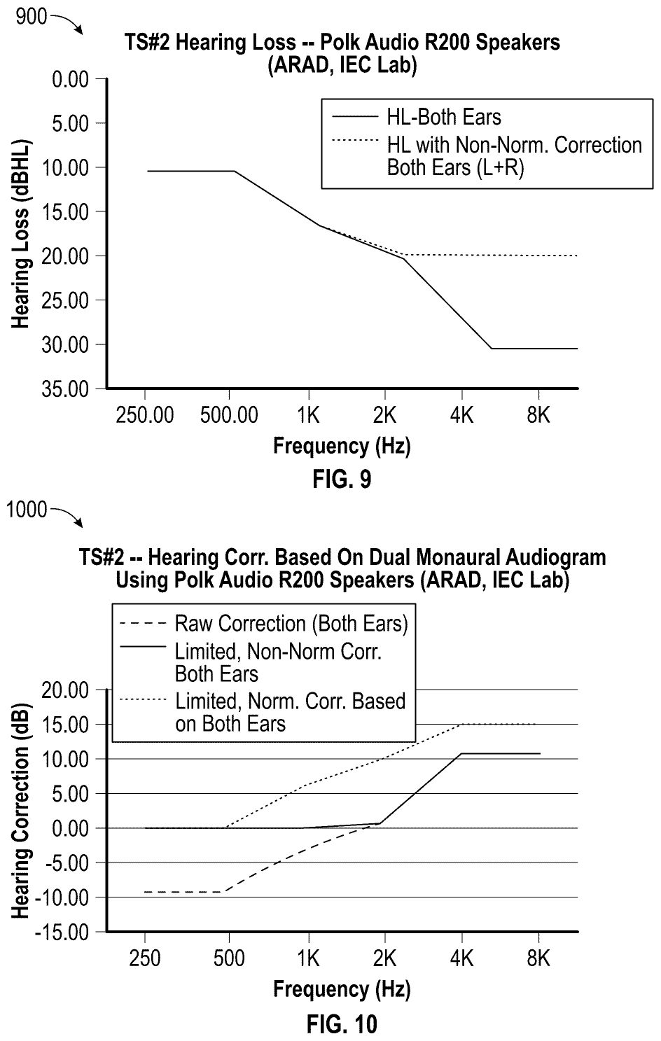

FIG. 9 is a plot or graph 900 illustrating the second test subject's dual monaural hearing loss (test subject #2 [TS #2]), as measured in a physical listening space with loudspeakers, and the expected hearing loss once the non-normalized correction (column (6) of Table 2) is applied. In FIG. 9, the solid line represents hearing loss in both ears (HL—both ears), whereas the broken or dotted line which overlaps the solid line from 250 Hz to 1 kHz represents hearing loss with non-normalized correction for both ears (L+R).

FIG. 10 provides a plot or graph 100 illustrating a comparison of hearing correction magnitude derived by the limited, normalized correction based on both ears (dotted line) and the limited, non-normalized correction based on both ears (solid line) described herein. Additionally, the “raw” correction for both ears (dashed line) is also included for reference. As indicated, the limited, normalized correction “curve” is the raw correction curve shifted in magnitude by 9.33 dB with a 15 dB limit imposed, in accordance with the values in Table 2.

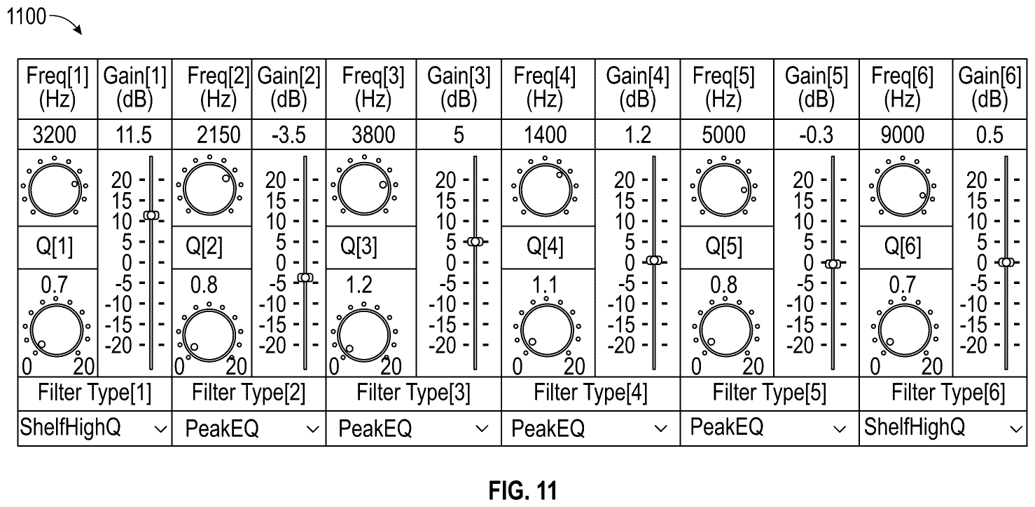

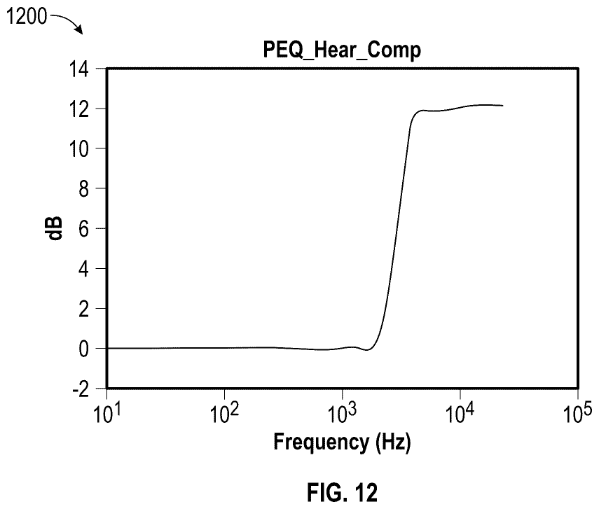

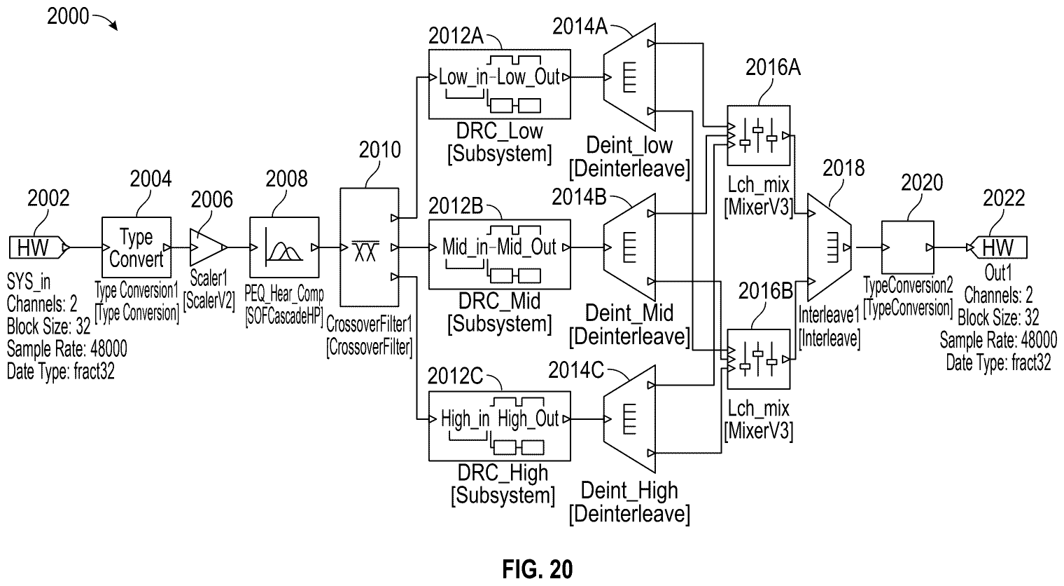

Alternative embodiments are useful for some applications. For example, with reference to the DSP signal flow diagrams of FIGS. 5A and 5B, the crossover or frequency dividing module 508 divides the incoming signal into three bands, but there may be a desire to have a greater number of frequency bands. DSP settings can be expressed as parametric equalizer settings as shown in settings graphic 1100 FIG. 11. The embodiment illustrated in FIG. 11 has six bands and different filter settings. In the embodiment of FIG. 11 the filter types differ (e.g., filter type 1 is a High Q shelf filter while filter type 2 is a peak EQ filter). In the system and method, the PEQ block may include as few as 2-3 and as many as 8-10 (or more) parametric EQ bands. As indicated in FIG. 11, six bands of PEQ are utilized in order to achieve the magnitude response curve 1200 illustrated in FIG. 12, which provides the targeted hearing correction for the test subject. It should be noted that several types of parametric filters are employed in the parametric filter block “PEQ_hear_comp” (e.g., 506 as seen in FIGS. 5A and 5B and 2008 in FIG. 20), including variable Q (quality factor) shelf filters and PeakEQ filters (variable frequency, gain and Q). Additionally, other types of filters such as high-pass, low pass are optionally employed as needed in order to achieve a target curve. Various types and combinations of filters can be used.

PEQ-hear-comp response plot 1200 of FIG. 12 illustrates the resulting magnitude response of the PEQ settings indicated in the settings graphic 1100 of FIG. 11. In accordance with the targeted “limited, non-norm” correction curve 1000 of FIG. 10, approximately 11-12 dB of gain from 4 kHz and above is achieved, while only minimal gain is applied below 2 kHz.

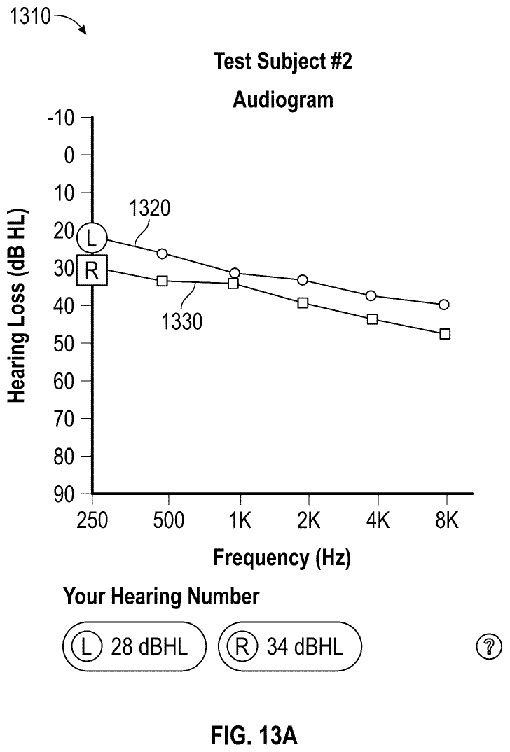

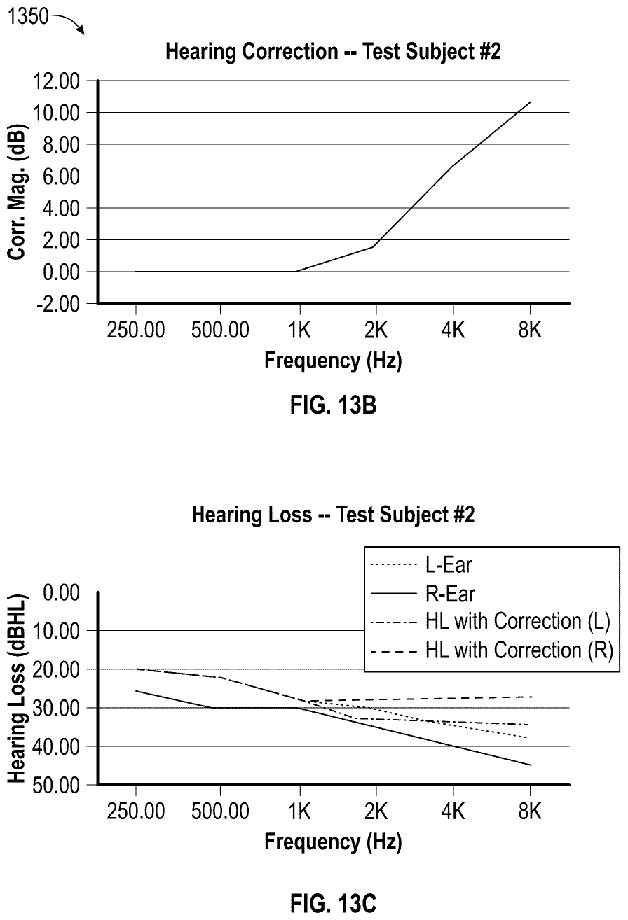

FIGS. 13A-13C relate to a system and method for corrective processing for a unique test subject (#2) using an all-in-one stereo (2.0) speaker (e.g., a Denon® Home DH250™ active loudspeaker system) according to an embodiment. FIGS. 13A-13C illustrate an embodiment of corrective processing for that all-in-one speaker (with test subject 2). Mimi audiogram 1310 for that test subject 2 is shown in FIG. 13A, and includes x-axis measurements at 250 Hz, 500 Hz, 1 kHz, 2 kHz, 4 kHz, and 8 kHz. Hearing loss is plotted on the y-axis in dBHL. Y-axis measurements start (at the bottom of the y axis) at 90 dB and decrease in increments of 10 dB to −10 dB. Audiogram 1310 may be acquired using an audiometry technique or service such as the Mimi™ (iOS) App or an online audiometer, such as found at www.onlineaudiometer.com. The “Hearing Number” for the left ear (representing the upper plot with lesser hearing loss in 1320 FIG. 13A) is 28 dB HL, while the “Hearing Number” for the right ear (representing the lower plot 1330 with greater hearing loss in FIG. 13A) is 34 dB HL. According to an embodiment, average HL values were computed for both ears over the 250 Hz to 8 kHz (six tone) measurement range (31 dB HL). As an alternative to using the average PT6 HL value of 31 dB, the lower PT6 hearing number of 28 dB HL associated the left ear may be used in the computation of the correction magnitude for each test frequency (see FIG. 13B). In the illustrated embodiment, correction magnitude was calculated as HLᵢ-HL_{avg}, unless HL_{avg} is greater than HLᵢ, in which case no correction was applied. For this equation, subscript “i” denotes frequency (i=250, 500, . . . 8kHz). The graph of FIG. 13B plots the correction for the test subject, with frequency (Hz) plotted on the x-axis and correction magnitude (dB) plotted on the y-axis. Correction was 0 dB for 250 Hz and approximately 10.00 dB at 8 kHz. The graph of FIG. 13C plots hearing loss, with frequency (Hz) plotted on the x-axis and hearing loss (dB HL) plotted on the y-axis. As can be seen, hearing loss with correction is particularly reduced (relative to no correction) at higher frequencies starting around 2 kHz up to 8 kHz.

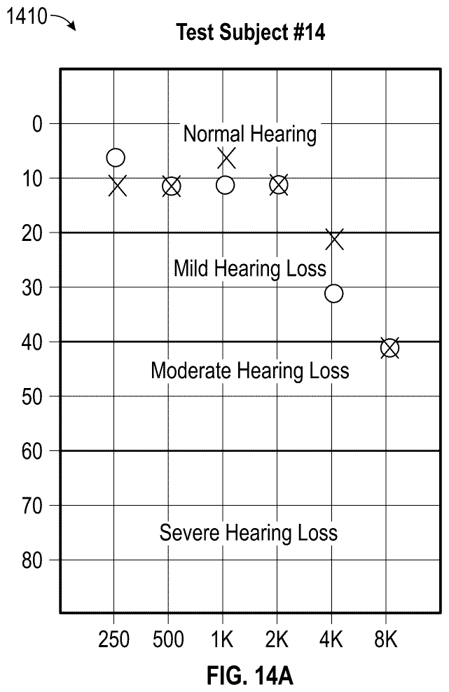

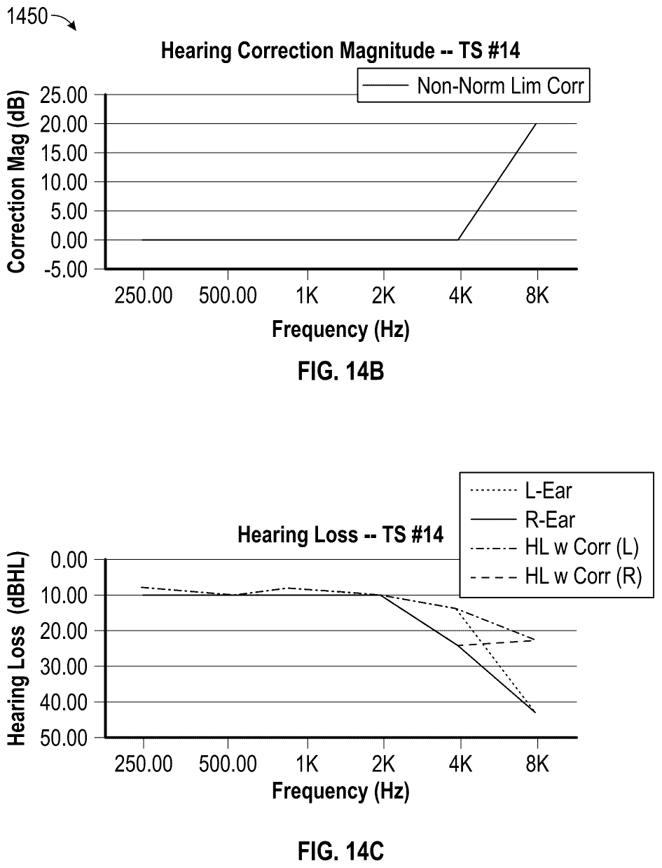

FIGS. 14A-14C relate to an embodiment for a Test Subject #14 (TS#14) comprising corrective processing for a 2.0 (stereo) all-in-one speaker system. Audiogram 1410 for TS #14 was first acquired (see FIG. 14A). The audiogram includes hearing loss (or sensitivity) measurements at 250 Hz, 500 Hz, 1 kHz, 2 kHz, 4 kHz, and 8 kHz. Hearing loss magnitude is plotted on the y-axis in dBHL. Y-axis measurements start at 80 dB and decrease in increments of 10 dB to 0 dB. The audiogram may be acquired using any technique, product, or service, including Mimi™ (iOS App.) or an online audiometer, such as found at www.onlineaudiometer.com. A composite audiogram was created from the lowest, best-ear HL at each test frequency, from which an average hearing loss value was computed. The average hearing loss HL_{avg} was calculated to be 15.5 dBHL. An audiogram was created that reflected the lowest hearing loss (i.e., the better ear, L or R) at each test frequency, as shown as FIG. 14A. “X”-plotted data points represent left ear values, and “O”-plotted data points represent right ear values. The correction magnitude for each frequency “i” was calculated as HLᵢ-HL_{avg}, unless HL_{avg} is greater than HLᵢ, in which case no correction was applied. If the correction magnitude exceeds the prescribed limit, it can be set to that gain level limit (e.g., 20 dB in this example). The graph of FIG. 14B plots the correction, with frequency (Hz) plotted on the x-axis and hearing correction magnitude (−5.00 dB to +25.00 dB) plotted on the y-axis. Correction was zero (0) for 250 Hz-4 kHz and approximately 20.00 dB at 8 kHz. The graph of FIG. 14C plots hearing loss with and without any correction applied, with frequency (Hz) plotted on the x-axis and hearing loss (dB HL) plotted on the y-axis.

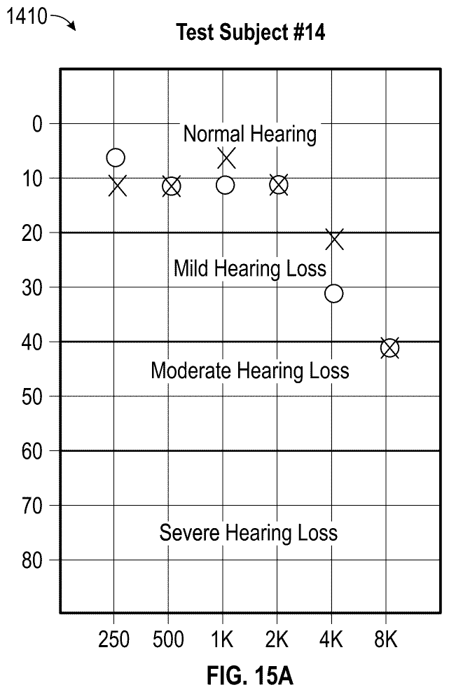

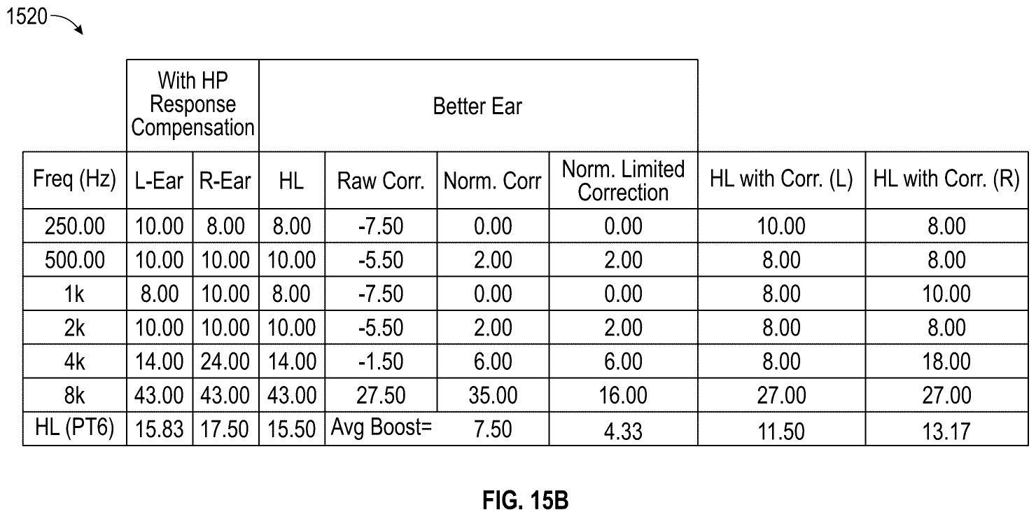

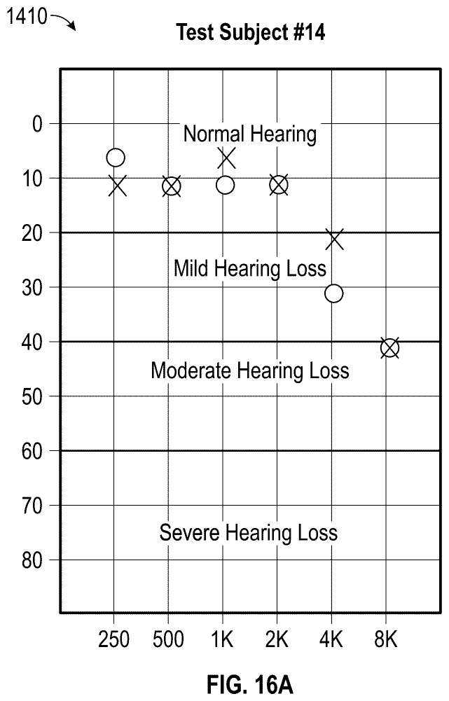

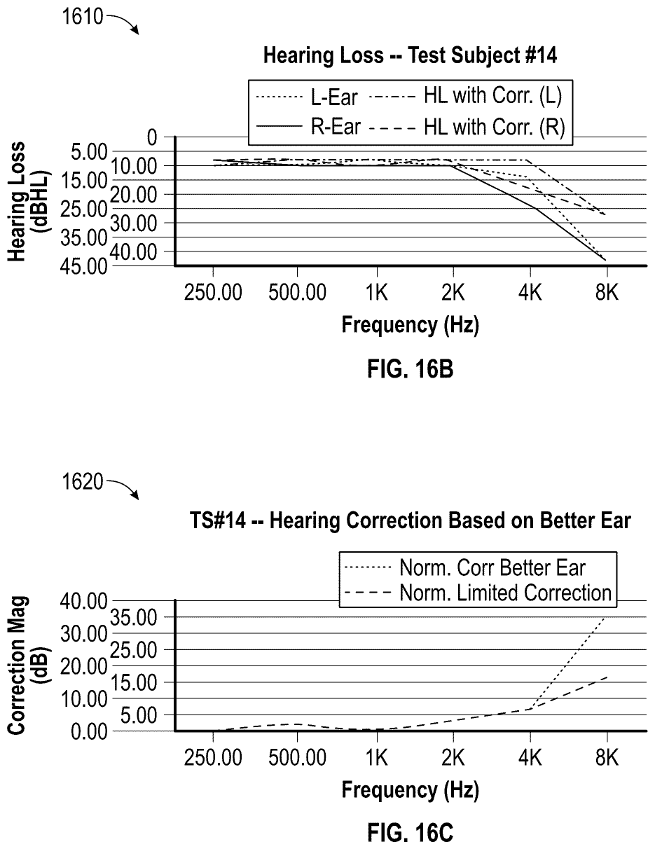

FIGS. 15A-15B and 16A-16C relate to an embodiment comprising corrective processing for loudspeakers. FIG. 15A shows an audiogram 1410 (for Test Subject #14 like that illustrated and described for the embodiment of FIG. 14A) that was acquired. As above, the audiogram may be acquired using any technique, product, or service, including Mimi (iOS) or an online audiometer, such as found at www.onlineaudiometer.com. In the embodiment of the method illustrated in FIGS. 15A-15B and 16A-16C, the next step is compensating for the headphone's frequency response (headphone calibration), which is applied to the data from audiogram 1410, with the results shown in the table 1520 of FIG. 15B. Next, hearing loss (HL) values were computed for both ears over the 250 Hz to 8 kHz range. The average hearing loss HL_{avg} was calculated to be 15.5 dBHL. An audiogram was created that reflected the lowest hearing loss (i.e., the better ear, L or R) at each test frequency, as shown in FIG. 15A. In this version of the method, headphone response compensation was applied to Online Audiometer results to Create a composite audiogram based on the lowest HL at each test frequency. Next, the average HL value was computed from the composite audiogram (HL_{avg}=15.50 dB), and then the “raw” correction was Normalized by adding 7.5 dB (lowest raw correction value). Next the correction limit was applied to the normalized correction (e.g., as shown, correction limit=16 dB).

FIGS. 16B and 16C provide two graphs 1610, 1620 plotting the data of the table 1520 of FIG. 15B for Test Subject 14. FIG. 16A shows the audiogram for the test subject 14. The graph 1610 has four traces plotting the hearing loss without correction for both (L and R) ears and hearing loss with correction for both ears. The graph 1610 illustrates that the user's or subject's hearing was corrected at 500 Hz and at 2.0 kHz and above in accordance with the normalized, limited correction shown in table 1520 of FIG. 15B.

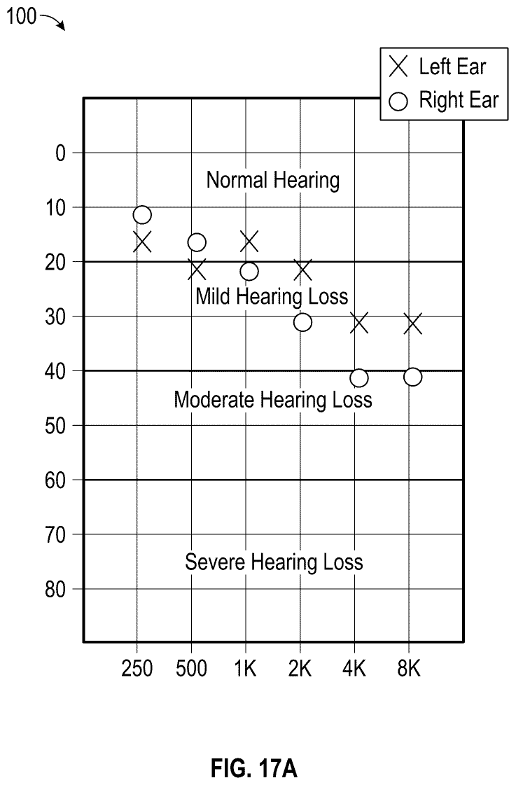

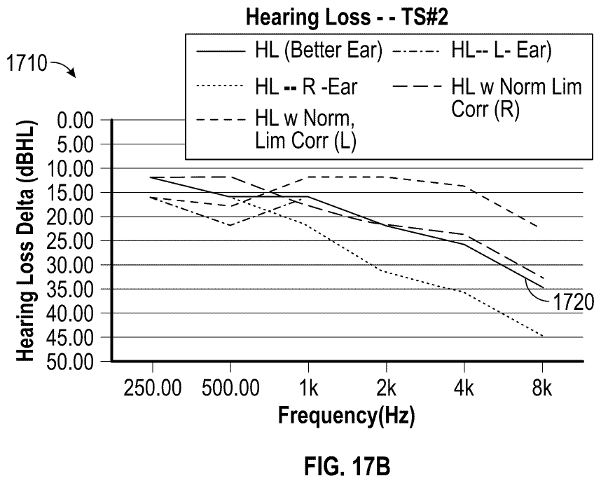

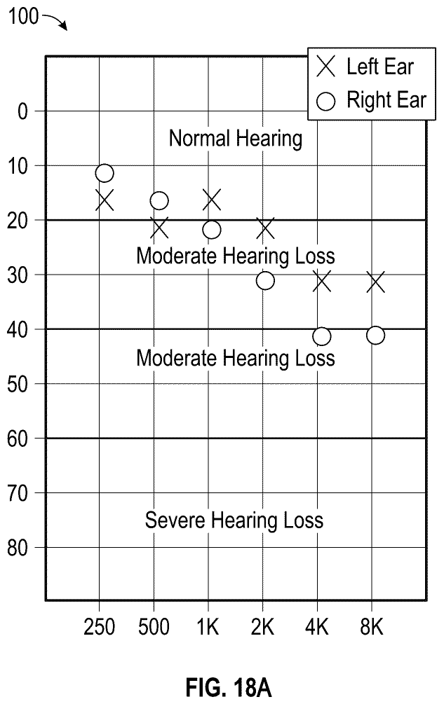

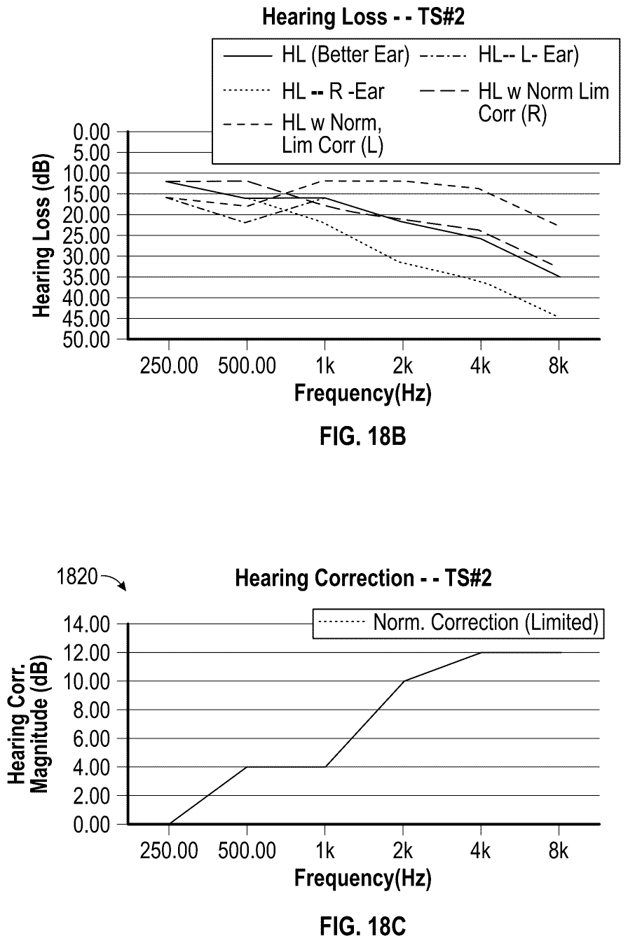

FIGS. 17A-17B and 18A-18C relate to another example embodiment. FIGS. 17A and 18A present audiogram 100 (for Test Subject 2, identical to that of FIG. 1), and hence relevant to the data in Table 1 above. In plot 1710 of FIG. 17B, hearing loss for the better ear is plotted with a solid line 1720. The other plotted lines in Hearing Loss plot 1710 illustrate the Hearing Loss for the Left Ear, Hearing Loss for the Right Ear, Hearing Loss for the Left Ear with normalized limited correction, and Hearing Loss for the Right Ear with normalized limited correction. FIG. 18B shows a plot of the hearing loss. FIG. 18C includes plot 1820 which illustrates Corrective processing (“Hearing Correction”) for loudspeakers derived from the “better” ear's hearing loss of FIGS. 17A-17B, where hearing correction is based on the “better” ear's HL (e.g., from a composite monaural audiogram) and the expected HL is based on the normalized, limited correction (where the maximum allowable boost is, in the illustrated example, 12 dB, as shown in plot 1820 of FIG. 18C).

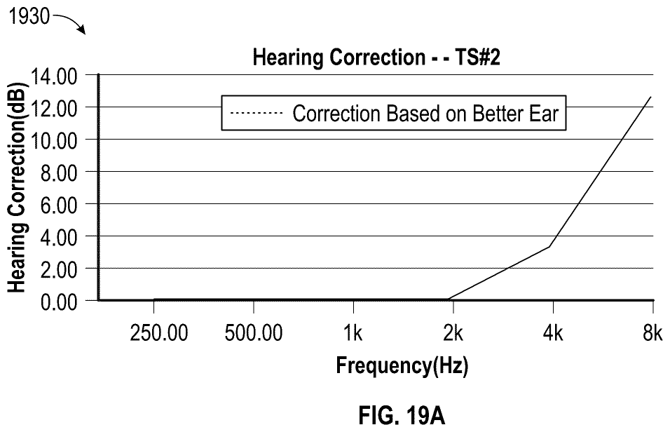

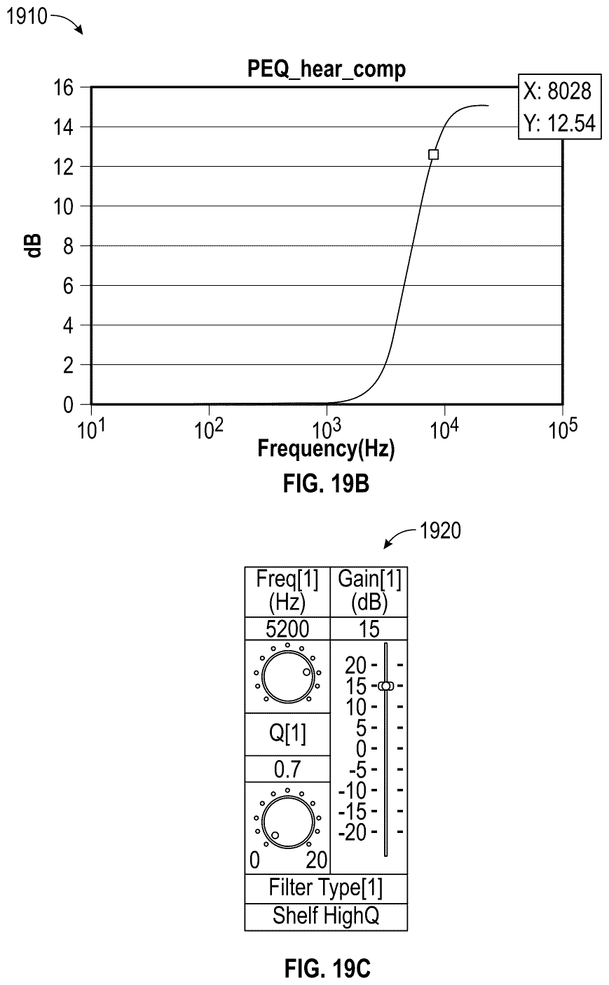

FIGS. 19A-19C relate to digital signal processing (DSP) settings 1910, 1920 for corrective processing for loudspeakers according to an embodiment. The corrective hearing magnitude response (EQ) target curve 1930 in FIG. 19A was derived from a test subject's audiogram (e.g., for Test Subject #2) in accordance with the non-normalized method in which a limit of 12 dB of gain has been imposed. Target curve 1930 for a hearing correction magnitude shaping may be substantially achieved by using a single parametric high-shelf filter whose settings are as indicated in DSP settings graphic 1920. While the upper bound of the targeted correction curve 1930 is 8 kHz, human hearing extends up to 20 kHz. As such, the applied magnitude correction for hearing compensation can likewise extend to 20 kHz which naturally aligns with the operating passband of the audio processing instruments employed for this purpose. Further, for individuals who experience high-frequency hearing loss, HL above 8 kHz-the upper limit of many common audiograms-may be presumed in some cases to exceed its measured 8 kHz level over the remaining 1.32 octave bandwidth comprising 8k-20 kHz. Therefore, in some embodiments, it may be preferable to “overshoot” the 8 kHz magnitude target to provide a moderate amount of “extra” boost for subjects that exhibit high-frequency hearing loss. As indicated in the magnitude response of the parametric shelf filter, 15 dB of boost is imposed above 15 kHz while at 8 kHz, the 12 dB target is substantially achieved.

FIG. 20 illustrates a DSP signal flow for use with an “all-in-one” two channel audio system such as a Denon Home DH250 active loudspeaker system, and the DSP signal flow can be applicable to various other speaker systems. The representative digital signal processing (DSP) signal flow illustrated in FIG. 20 shows three frequency bands (low, medium, and high). The system 2000 shown in FIG. 20 provides an embodiment representing the signal processing applied to program material for hearing compensation/correction purposes. The system 2000 represents an example embodiment of a sequence in which constituent processes of signal flow are implemented. The associated digital signal processing (DSP) settings reflect the individual's hearing profile.

In FIG. 20, the personal hearing correction EQ (PEQ_hear_comp) may be set to match the derived correction EQ. Also, band dependent compression for limiting boost (DRC_low, DRC_mid, DRC_high) at higher sound levels may be applied, for example, where only minimal corrective boost is appropriate or desired. Also, the hearing correction processing may include a “loopback” from the loudspeaker system's master volume control in order to ensure proper engagement of the band dependent compressors and post processing gain (e.g., after module 2020).

During development, several comparative listening sessions were conducted and the participating test subjects' subjective listening results were noted for the example embodiments. It should be understood that these subjective listening results are not necessarily applicable to all embodiments described herein. The following observations form part of the present disclosure's development process:
1. For listeners with substantial differences in their binaural hearing (e.g., one ear is much “better” than the other), basing correction on the average of the two ears tends to result in an overcorrection. For such listeners, test subjects prefer when hearing correction is based on the better ear. If a high level of interaural crosstalk cancellation is achieved, then a separate correction could be applied to each ear as needed.
2. For some test subjects, the difference between uncorrected and corrected hearing loss was subtle, even barely noticeable, at least with some program material.
3. Experienced listeners immediately noticed the difference between uncorrected and corrected hearing loss, but did not necessarily prefer the processed over the “pure” unadulterated (uncorrected) program material.
4. For some listeners, reducing the correction magnitude from the computed “ideal” (corrected) hearing loss resulted in a more pleasing, spectrally balanced sound.
5. Hearing correction processing has been applied to both an all-in-one active speaker (such as Denon™ DH250) and to a pair of hi-fi bookshelf speakers (such as Polk™ R200 50th Anniversary Edition) driven by a Denon™ AVR. The Polk™ R200 are more revealing of the upstream hearing-correction processing, such that “dialing back” the correction magnitude may be appropriate.

### Embodiment Including Smartphone App

One of many possible embodiments shall include a means of accessing a user's previously acquired hearing profile, whether OAE-based or a hearing threshold audiogram, so as to determine the appropriate, personalized hearing correction processing settings for the host loudspeaker-based audio system. A smartphone application, developed for this purpose, can provide a user interface portal to the hearing-correction enabled loudspeaker system. Denon's HEOS system, an audio networking and control system which offers a means of selecting audio systems, including individual all-in-one systems, stereo loudspeakers and complete home theatre systems for playing selected audio program material, is well suited to support personalized hearing correction over loudspeakers as it shall be capable of storing and recalling a user's hearing profile and communicating playback preferences with HEOS enabled audio systems. Various audio systems and other communication systems can be used to store and transmit hearing profile or audiogram information, which can be used by audio devices or systems to provide hearing-corrected audio using the principles disclosed herein.

Some embodiments can include a system and method for controlling the selected hearing-corrective loudspeaker system by, for example, use of a smartphone application (iOS or Android), an IR, RF or Bluetooth enabled remote control which would necessarily include a complementary IR/RF/BT receiver on the active soundbar/loudspeaker or AVR end, and/or an on-screen display on the active soundbar/loudspeaker itself, the AVR and/or on a connected video monitor. Further, in the absence of a previously acquired hearing profile, the hearing-corrective loudspeaker system's user-interface may optionally provide a means of acquiring the user's hearing profile by the dual-monaural loudspeaker method in which the loudspeakers themselves emit test tones for ascertaining the user's hearing thresholds for each test tone or by a binaural headphone/earbud method (OAE or hearing threshold-based audiogram).

It will be appreciated by persons of skill in the art that a loudspeaker system driven by a corrected audio output signal adjusted for playback in a user's listening space would be applicable to not only domestic or office environments (e.g., as in FIG. 7) but also automotive interiors. As such, in an automotive interior (e.g., as described and illustrated in commonly owned U.S. Pat. No. 11,838,740 entitled “Automotive audio system and method with tri-polar loudspeaker configuration and floating waveguide equipped transducers in an automotive headrest” the entire disclosure of which is incorporated herein by reference), a corrected automotive audio loudspeaker system may be driven by a corrected audio output signal adjusted for playback in an automotive interior space at a selected position (e.g., the driver's seat).

Hearing corrective signal processing parameters, generally including magnitude shaping, multiband and multi-stage compression, may optionally be set in accordance with a user's hearing profile (as described above). Yet some users may prefer to implement hearing correction that is moderated with respect to their nominal, ideal settings. Such a “low” correction setting would include, in part, lower gain settings in magnitude shaping, lower DRC threshold settings and higher DRC compression ratios. Similarly, others may prefer more extreme, or enhanced, hearing correction settings which generally would reflect, with respect to their nominal, ideal settings, higher gain magnitude shaping, higher compression thresholds and lower compression ratios for the DRC preceding the limiter in the multi-band, serial compressor sequence. Example settings, numerical or relative, for low/default/high (or “enhanced”) hearing correction are shown in Table 3.

TABLE 3
Example, relative settings for low, default and high (enhanced) hearing correction

| | Magnitude | Magnitude | | DRC | DRC | |
|---|---|---|---|---|---|---|
| Hearing | Shaping | Shaping | DRC | compression | attack/ | Expansion/ |
| Correction | Gain | Limit | threshold | ratio | release | Compander |
| Low | 3-6 dB | 6-10 dB | 6-10 dB | 5:1 up | Shorter/ | No |
| | lower than | | lower | to10:1 | longer | |
| | “ideal | | than | | | |
| | | | “ideal” | | | |
| Default | Ideal, per | 12-20 dB | “ideal” | 2:1 to 3:1 | Moderate, | No |
| | processing | | settings | (as determined | for optimal | |
| | algorithm | | | to be optimal) | effect | |
| High | 3-6 dB | 18-24 dB | 3-10 dB | 1:1 up to | Longer/ | Yes, as needed |
| (“enhanced”) | higher | | higher | 1.5:1 | shorter | for achieving |
| | than | | than | | | targeted effect |
| | default | | “ideal” | | | |

### Age Related Hearing Loss (Presbycusis)

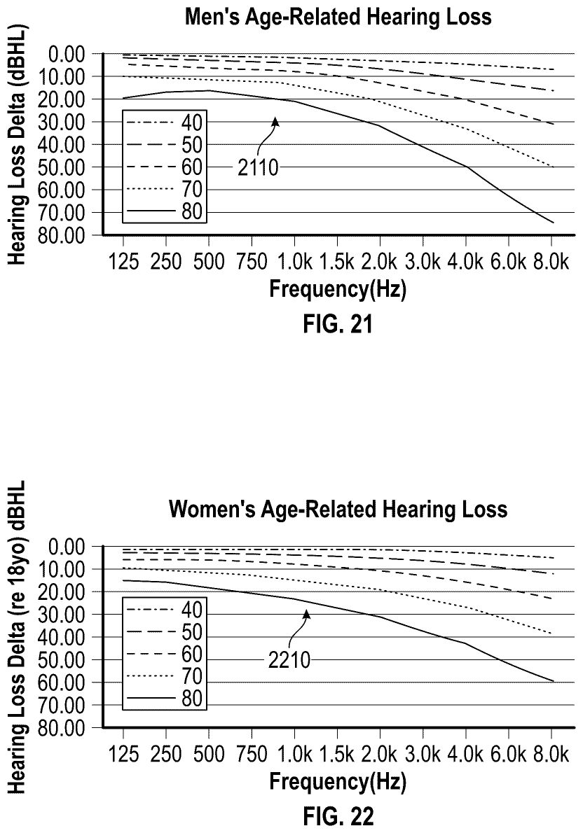

While personalized hearing profiles ideally address an individual's hearing loss, acquiring a specific or particular subject's or user's hearing profile does involve conducting a hearing test which may be inconvenient or burdensome for a given user. Some users may prefer to implement a generalized hearing correction based on their age and other factors such as gender (male or female) and known hearing loss. Those users or subjects may instead select an approximation, and thereby acquire an estimated or approximate user hearing profile. For the description below, FIG. 21 illustrates Age-Related Hearing Loss (Presbycusis) for Men aged 40-80 years old, relative to an otologically normal 18 year old man (normal, healthy hearing). Per ISO 7029, an “otologically normal” person is defined as someone “in a normal state of health who is free of all signs or symptoms of ear disease, free of obstructing wax in the air canal and who has no history of undue exposure to noise, exposure to potentially ototoxic substances or familial hearing loss”. FIG. 22 illustrates Age-Related Hearing Loss (Presbycusis) for Women aged 40-80 years old, relative to an otologically normal 18 year old woman.

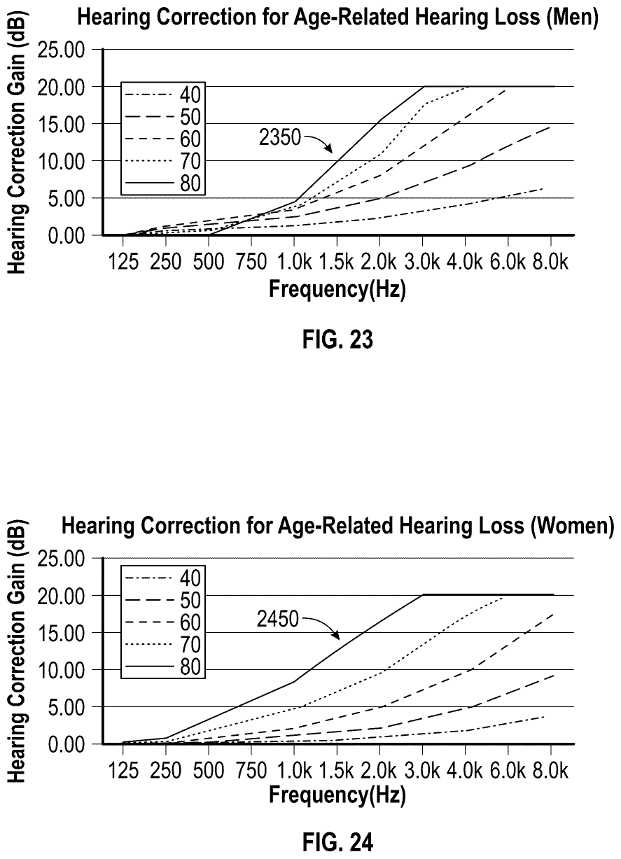

The hearing loss audiograms (2110, 2210) plotted in FIGS. 21 and 22 are used to determine appropriate hearing corrections (2350, 2450). FIG. 23 illustrates Hearing correction for age-related hearing loss (normalized and limited to 20 dB), for men aged 40-80 years old (2350), and FIG. 24 illustrates Hearing correction for age-related hearing loss (normalized and limited to 20 dB), for women aged 40-80 years old (2450). FIGS. 21 and 22 illustrate hearing loss for men and women respectively versus age in reference to a same gender otologically normal 18-year-old. The data from which these Figures are derived are shown in TABLE 4. These data have been computed using a formula presented in ISO 7029:

$$Δ {H}_{md , Y} = {{α}_{md} ( Y - 18 )}^{{β}_{md}} . \qquad ( 1 )$$

This formula estimates the hearing loss deviation (in dBHL) relative to a same gender otologically normal 18 year-old. In Table 4, The parameters α_{md} and β_{md} are dimensionless quantities, while Υ is the lower limit of 18 years old for the age range over which Formula (1) is valid.

TABLE 4
Table - Values of α_{md} and β_{md} in Formula (1)

| | | |
|---|---|---|
| Frequency | α_{md} | β_{md} |

| Hz | Males | Females | Males | Females |
|---|---|---|---|---|
| 125 | 2.50 × 10⁻⁶ | 6.16 × 10⁻⁴ | 3,841 | 2,451 |
| 250 | 1.39 × 10⁻⁴ | 3.98 × 10⁻⁴ | 2,832 | 2,568 |
| 500 | 4.59 × 10⁻⁴ | 2.61 × 10⁻⁴ | 2,537 | 2,708 |
| 750 | 5.70 × 10⁻⁴ | 2.25 × 10⁻⁴ | 2,512 | 2,775 |
| 1 000 | 7.02 × 10⁻⁴ | 2.21 × 10⁻⁴ | 2,494 | 2,805 |
| 1 500 | 1.09 × 10⁻³ | 2.53 × 10⁻⁴ | 2,446 | 2,813 |
| 2 000 | 1.56 × 10⁻³ | 3.12 × 10⁻⁴ | 2,404 | 2,792 |
| 3 000 | 2.54 × 10⁻³ | 4.88 × 10⁻⁴ | 2,350 | 2,728 |
| 4 000 | 3.40 × 10⁻³ | 7.37 × 10⁻⁴ | 2,325 | 2,660 |
| 6 000 | 4.53 × 10⁻³ | 1.47 × 10⁻³ | 2,315 | 2,539 |
| 8 000 | 5.06 × 10⁻³ | 2.53 × 10⁻³ | 2,328 | 2,439 |

Of note, men's high frequency hearing loss is substantially greater than women's relative to same sex 18 year olds. For example, at 4.0 kHz a 60 year-old man may experience approximately 20 dBHL of hearing loss relative to an 18 year old man in comparison to a 60 year-old women whose expected hearing loss would be only 15 dBHL at that frequency (relative to an 18 year old woman) in accordance with FIGS. 21 and 22. In a manner and method that's analogous to those by which personalized hearing correction processing parameters are determined for an individual whose audiogram is available or acquired, hearing correction processing parameters for presbycusis can reflect an inverse of the individual's expected hearing loss on the basis of their age and gender. Further, in accordance with the normalization and limiting routines as presented herein for application to personalized hearing profiles, compensation for age-related hearing loss shall be normalized such that the compensation gain applied at 125 Hz or lower frequencies shall be set to 0 dB. Additionally, corrective gain can be limited, such as to 12-20 dB in order to maintain adequate dynamic range and low distortion. Most importantly, as stated previously herein, studies conducted by the inventor indicate that applying excessive corrective gain to audio program material when reproduced over loudspeakers can result in unpleasant, unnatural sound quality due to distortion artifacts.

An example embodiment in which age and gender related hearing correction may be established first, the user shall be asked to provide their age and gender. Then the user shall be presented with an option for (1) providing or enabling access to their previously acquired hearing profile, (2) measuring their hearing via the (2a) dual-monaural loudspeaker method, or (2b) a headphone-based method or (3) applying hearing correction settings based on their age and gender. When option (3) is selected, the user's age and gender related hearing profile, e.g., stored in “look-up tables” which tabulate all of the associated processing values, including magnitude shaping coefficients and DRC parameters, shall be implemented in accordance with the user's age and gender. Employing the user's actual hearing profile (options 1 and 2), is preferred in some cases over the age/gender-based method since the former ones more accurately reflect the individual's hearing acuity. Finally, regardless of which option the user selects, their age and gender can be recorded assuming that the user complies with this request. Then, their acquired profile can be compared to their expected profile as a means of confirming the validity of the hearing test. Unless the user has suffered profound hearing loss due to noise exposure, disease or some other reason, their hearing profile ought to substantially agree with their age and gender related profile. ISO 7029 provides a method of estimating the range about mean hearing loss on the basis of age and gender which may be employed in order to set reasonable bounds for the deviation between the user's measured hearing and their expected audiogram on the basis of age/gender. Excessive deviations suggest that the user's acquired audiogram is invalid and should be reacquired or that the user's hearing actually differs greatly from the expected mean hearing loss, possibly due to excessive noise exposure or medical (disease, congenital) reasons. There are a number of possible ways of handling such discrepancies, including a protocol for repeating some or all of the hearing test in order to confirm or invalidate the previously acquired results or relying on the age/gender related profile if conducting a valid hearing test proves to be impossible or difficult.

### Alternative Signal Flow/Algorithm Hearing-Correction Process

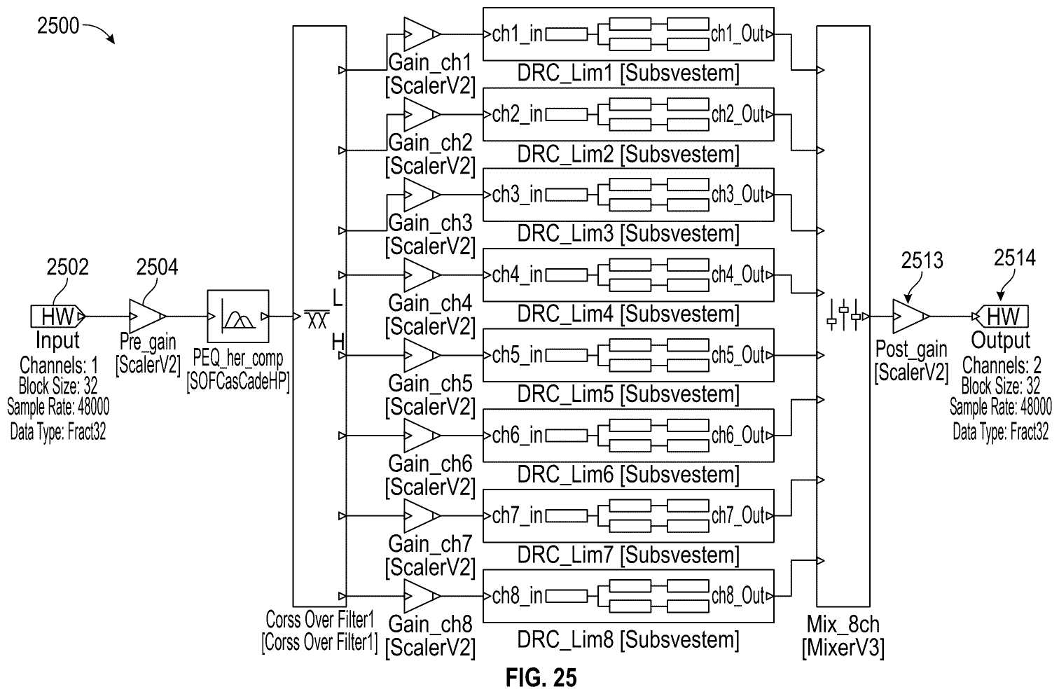

As another method for generating hearing correction, the hearing loss values for an individual test subject that have been determined by the methods described herein may be employed directly in the hearing correction signal flow/process as illustrated in FIG. 25. DSP system 2500 may be contrasted by DSP system 500 and 600 shown in FIGS. 5A and 5B by an absence of any parametric equalization blocks (e.g. 506). Instead, the audio signal is divided in crossover block 2508 into n+2 (e.g., eight) sub-bands where n corresponds to the number of test frequencies in an audiogram (e.g., 100). As illustrated in Table 5, these sub-bands comprise the audiogram test frequency passband (250-8.0 kHz, for example) plus the frequency ranges below and above such that the entire audio passband of human hearing, 20 Hz-20 kHz, is covered.

TABLE 5

| | 2 | 3 | 4 | | | |
|---|---|---|---|---|---|---|
| | freq. sub-bands (Hz) | | | | 6 | 7 |
| | | center | | 5 | Hearing | Norm/Lim |
| 1 | lower | (audiogram | upper | XO | Loss | Correction* |
| Band | bound | test freq) | bound | “cutoff” | (dBHL) | (dB) |
| 1 | 20 | N/A | 175 | 175 | NA | 0 |
| 2 | 175 | 250 | 350 | 350 | 12 | 0 |
| 3 | 350 | 500 | 700 | 700 | 16 | 4 |
| 4 | 700 | 1.0k | 1.4k | 1.4k | 16 | 4 |
| 5 | 1.4k | 2.0k | 2.8k | 2.8k | 22 | 10 |
| 6 | 2.8k | 4.0k | 5.6k | 5.6k | 26 | 14 |
| 7 | 5.6k | 8.0k | 11.2k | 11.2k | 35 | 16 |
| 8 | 11.2k | N/A | 20k | NA | NA | 16 |

*Lim = 16 dB

In both FIG. 25 and Table 5, the audiogram test frequencies comprise bands 2-7 (inclusive) and their one-octave passbands are as indicated in Table 5. In the method, the gain applied to each of the sub-bands 2-7 can be normalized and limited hearing correction can be determined by the processes described herein, and these values for a representative hearing correction test subject are illustrated in Table 5. That is, the gain values G2, G3. . . . G7 for the amplifier blocks illustrated in FIG. 25 can address the test subject's hearing loss values, in accordance with the “best ear” composite audiogram. In particular, for the six-frequency audiogram presented herein, gain values G2, G3, . . . G7 shall substantially or exactly match the test subject's normalized and limited hearing correction based on their best-ear composite audiogram after application of Rules (1) and (2) described above. Again, a first parameter or Rule One is that no attenuation is applied in the hearing correction when the subject's hearing loss is negligible or zero for a particular audiogram test frequency.

A second parameter or Rule Two can ensure that the hearing correction system operates within its own dynamic range and that risks of clipping distortion are minimized, reduced, or impeded by limiting corrective gain to a pre-selected maximum value (e.g., 12-20 dB). In the example of Table 5 and FIG. 25, that Rule two maximum gain is limited to 16 dB. The two gain values G1 and G8 associated with bands 1 and 8 respectively which bracket the audiogram test bands' passband of 175 Hz-11.2 kHz substantially match their adjacent sub-bands 2 and 7 respectively as indicated in Column 7 of Table 5. Further, as shown in FIG. 25, dedicated multi-stage DRC/limiters operate on each of the eight sub-bands. The parametric compressor values for DRC/Lim1, 2, etc. for each sub- band 1, 2 etc. depend generally on the upstream gain value (e.g. G1, G2, etc.). For example, DRC/Lim6, when following a relatively large gain value for the upstream gain block G6 (e.g. G6=14 dB) are set such that its threshold is set to a relatively low value (e.g. −50 dB). Downstream from the bank of eight DRC/Limiters in FIG. 25 is a summing block 2512 in which attenuation is set to be the same for each input such that a net of unity gain is achieved. The output of summing block 2512 is corrected output 2514.

### Correcting Loudspeaker Playback in Automotive Interiors

The concept of hearing loss compensation (hearing correction) over loudspeakers is applicable to not only home, residential environments and other living spaces but also to automotive interiors. The audio processing parameters associated with compensating for an individual's hearing loss, such as determined on the basis of their audiogram acquired over headphones (e.g., otoacoustic emissions (OAE) method or tonal audiogram) or loudspeakers in-situ as described herein, or otherwise, are applied to an automotive loudspeaker system for this purpose. Further, it is possible via beam-steered arrays, highly directional loudspeaker systems that utilize horns or waveguides, or close-proximity loudspeakers (located within an automotive seat's headrest) and other methods to substantially isolate an occupant for purposes of delivering audio programs to selected individuals or micro-zones within the automotive passenger compartment (see, e.g., commonly owned U.S. Pat. No. 11,900,909 entitled “System and method for providing a quiet zone” which describes a system and method for determining and then generating an active noise cancellation signal, the entire disclosure of which is incorporated by reference for all that it discloses). Additional beamforming and other details that can be applied to the systems and methods disclosed herein are described in U.S. patent application Ser. No. 18/903,481, which is incorporated by reference herein for all that it discloses. Hearing corrected audio can be directed to each or some subset of the vehicle's occupants in this manner. It will be appreciated by persons of skill in the art that a loudspeaker system driven by a corrected audio output signal adjusted for playback in a user's listening space which can be a pre-defined automotive interior (e.g., of the type described and illustrated in U.S. Pat. No. 11,838,740 entitled “Automotive audio system and method with tri-polar loudspeaker configuration and floating waveguide equipped transducers in an automotive headrest” the entire disclosure of which is incorporated herein by reference), an automotive audio loudspeaker system may be driven by a corrected audio output signal adjusted for playback in an automotive interior space at a selected position (e.g., the driver's seat).

Applications and advantages identified herein may be applicable to example embodiments. The scope of the disclosure, however, is not necessarily restricted to system and methods that encompass such advantages.

Embodiments of the systems, products, and methods of example embodiments will help those persons who suffer from moderate hearing loss to better experience any reproduced program material, such as music, movie soundtracks, spoken word (podcasts) etc. In conjunction with the identification of a personal hearing profile, such as a previously determined personal hearing profile, example embodiments disclosed herein automatically implement corrective audio processing.

In the context of software, the operations described herein represent non- transitory program code (e.g., computer-executable instructions, datastores, etc.), which may be stored on one or more tangible, computer-readable storage media that, when executed by one or more processors, perform the recited operations. Generally, computer-executable instructions include routines, programs, objects, components, data structures, and the like that perform particular functions or implement particular abstract data types. The order in which the operations are described is not intended to be construed as a limitation, and any number of the described operations can be combined in any order and/or in parallel to implement the process.

The term “module” refers broadly to locally executed software, software executed in the cloud, hardware, or firmware components (or any combination thereof). Modules are typically functional components that can generate useful data or other output using specified input(s). A module may or may not be self-contained. An application program (also called an “application”) may include one or more modules, or a module may include one or more applications. Generally, modules may include programs, components, objects, routines, logic, data structures, and so on that perform and implement particular tasks. A task may be practiced in distributed cloud computing environments, where tasks are performed by remote processing devices that are linked through a communications network. Modules may be located in a local computer system storage medium/media and/or remote computer system storage medium/media. Modules generally carry out the functions and/or methodologies of embodiments.

Modules may be stored in, for example, memory, operating systems, one or more application programs, other program modules, and program data. The memory may include computer-readable storage media (“CRSM”), which may be any available physical media accessible by the processor to execute instructions stored on the memory. In an implementation, CRSM may include random access memory (“RAM”), flash memory and/or cache memory. In other implementations, CRSM may include, but is not limited to, read-only memory (“ROM”), electrically erasable programmable read-only memory (“EEPROM”), or any other medium which can be used to store the desired information and which can be accessed by the processor.

Computer programs are stored in memory, which may be physically present on the hearing protection device or an external device/system in communication with the hearing protection device. Computer programs may also be received via a communication interface. Such computer programs, when run, enable the computer system to perform the features of the present embodiments as discussed herein. In particular, the computer programs, when run, enable the processing unit to perform the features of the computer system. Accordingly, such computer programs represent controllers of the computer system. For example, a storage system may be provided for reading from and writing to a non-removable, non-volatile magnetic media (e.g., a hard drive) or other memory. A magnetic disk drive for reading from and writing to a removable, non-volatile magnetic disk (e.g., a disk), and an optical disk drive for reading from or writing to a removable, non-volatile optical disk such as a CD-ROM, DVD-ROM or other optical media can be provided. In such instances, each can be connected to bus by one or more data media interfaces.

Terms “computer program medium,” “computer usable medium,” and “computer-readable medium” are used to generally refer to media such as main memory, including RAM, cache, and storage system, such as a removable storage drive and a hard disk installed in a hard disk drive. Computer-readable storage medium can be a tangible device that can retain and store instructions for use by an instruction execution device. The computer-readable storage medium may be, for example and without limitation, an electronic storage device, a magnetic storage device, an optical storage device, an electromagnetic storage device, a semiconductor storage device, or any suitable combination of the foregoing. A computer-readable storage medium, as used herein, is to be construed as non-transitory, and is not to be construed as being transitory signals per se.

While particular embodiments have been shown and described, it will be understood to those skilled in the art that based upon the teachings herein, changes and modifications may be made without departing from its broader aspects. Therefore, the appended pseudo claim statements are to encompass within their scope all such changes and modifications as are within the true spirit and scope of the embodiments. Furthermore, it is to be understood that the embodiments are solely defined by the appended pseudo claim statements. It will be understood by those with skill in the art that if a specific number of an introduced pseudo claim statement element is intended, such intent will be explicitly recited in the pseudo claim statements, and in the absence of such recitation no such limitation is present. For non-limiting example, as an aid to understanding, the following appended pseudo claim statements may contain usage of the introductory phrases “at least one” and “one or more” to introduce pseudo-claim statement elements. However, the use of such phrases should not be construed to imply that the introduction of a pseudo claim statement element by the indefinite articles “a” or “an” limits any particular pseudo claim statement containing such introduced pseudo claim statement element to the embodiments containing only one such element, even when the same pseudo claim statement includes the introductory phrases “one or more” or “at least one” and indefinite articles such as “a” or “an”; the same holds true for the use in the pseudo claim statements of definite articles. As used herein, the term “and/or” means either or both (or any combination or all the terms or expressed referred to).

Embodiments described herein may be, among other things, a system, a computer program product, and a method. Selected aspects and features of example embodiments described herein may take the form of an entirely hardware embodiment, an entirely software embodiment or an embodiment combining software and/or hardware aspects that may all generally be referred to herein as a “circuit,” “module” or “system.” Furthermore, aspects of example embodiments may take the form of computer program product embodied in a computer readable storage medium (or media) having computer readable program instructions thereon for causing a processor to carry out aspects of the present embodiments.

Aspects are described herein with reference to flowchart illustrations and/or block diagrams of systems, computer program products, and methods according to embodiments. Each block of the flowchart Illustrations, block diagrams, and combinations of blocks can be implemented by computer readable program instructions. The computer readable program instructions may be provided to a processor to produce a machine, such that the instructions, which are executed via the processor, implement the functions/acts specified in the flowcharts and/or block diagram block or blocks. These computer readable program instructions may also be stored in memory, such as described herein, such that the computer readable storage medium having instructions stored therein comprises an article of manufacture including instructions which implement aspects of the function/act specified in the flowchart and/or block diagram block or blocks.

The flowcharts and block diagrams in the Figures illustrate examples of the architecture, functionality, and operation of certain implementations of systems, computer program products, and methods in accordance with certain embodiments. In this regard, each block in the flowchart or block diagrams may represent a module, segment, or portion of instructions, which comprises one or more executable instructions for implementing the specified logical function(s). In some alternative implementations, the functions noted in the block may occur in a different order or sequence than that noted in the figures. Further, two or more blocks shown in succession may, in fact, be executed substantially concurrently, or the blocks may sometimes be executed in the reverse order. In other implements, one or more blocks may be omitted, and/or additional blocks not shown in the accompanying Figures may be included within the scope of the systems, computer program products, methods, and other embodiments described herein.

Persons of skill in the art will appreciate that the present disclosure makes available a loudspeaker system and method to measure a specific user's hearing and implement compensatory processing to address the user's hearing deficiencies. Hearing acuity may be determined by several different techniques, including but not limited to (i) headphone techniques in which hearing frequency dependent thresholds are determined; (ii) a loudspeaker system set up as intended for use in an acoustic space to emit tonal stimuli, (iii) inducing, acquiring and interpreting otoacoustic emissions, also known as the human hearing system's natural response to acoustic stimuli. According to an embodiment, once the hearing profile is determined, for example by any of these techniques, appropriate signal processing parameters are implemented.

The corresponding structures, materials, acts, and equivalents of all means or step plus function elements in the examples herein are intended to include any structure, material, or act for performing the function in combination with other example elements as specifically recited. The description of the present embodiments has been presented for purposes of illustration and description, but is not intended to be exhaustive or limited to the embodiments in the form disclosed. Many modifications and variations will be apparent to those of ordinary skill in the art without departing from the scope and spirit of the embodiments. The embodiments were chosen and described in order to best explain the principles of the embodiments and the practical application, and to enable others of ordinary skill in the art to understand the embodiments for various embodiments with various modifications and combinations with one another as are suited to the particular use contemplated. Accordingly, the scope of protection of the embodiment(s) is limited only by the claims and their equivalents.

Having described example embodiments of a new and improved apparatus and method, it is believed that other modifications, variations and changes will be suggested to those skilled in the art in view of the teachings set forth herein. It is therefore to be understood that all such variations, modifications and changes are believed to fall within the scope of the present disclosure.

### Example Embodiments

Embodiment 1. A hearing correction loudspeaker system, comprising:
a loudspeaker system configured for use in a listening space (e.g., as illustrated in FIG. 7), said loudspeaker system being responsive to a specific user's hearing and a hearing correction system includes signal processing which implements compensatory or corrective signal processing are provided to address the user's hearing deficiencies; wherein said signal processing is programmed with appropriate signal processing parameters which are derived and implemented to generate a corrected audio output signal adjustment (e.g., 1000, 1360, 1450 comprising a selected plurality of correction EQ signals each covering respective frequency sub-bands (“correction EQ frequency bands”);
wherein administered hearing test results 100, 1310, 1410 comprise a plurality of hearing loudness threshold levels at respective test frequencies which the test subject detects in response to hearing audio stimuli at those test frequencies;
   wherein hearing correction system's corrected audio output (e.g., 1350, 1450) is generated from corrected loudness values at selected correction frequency sub-bands (“correction EQ frequency bands”);
and the loudspeaker system is driven by the corrected audio output signal which is adjusted for playback in the specific user's listening space (e.g., in a home or office).

Embodiment 2. An automotive hearing correction loudspeaker system, comprising:
a loudspeaker system configured for use in an automotive interior listening space, said loudspeaker system being responsive to a specific user's hearing and a hearing correction system includes signal processing which implements compensatory or corrective signal processing are provided to address the user's hearing deficiencies;
wherein said signal processing is programmed with appropriate signal processing parameters which are derived and implemented to generate a corrected audio output signal adjustment (e.g., 1000, 1360, 1450 comprising a selected plurality of correction EQ signals each covering respective frequency sub-bands (“correction EQ frequency bands”);
wherein administered hearing test results 100, 1310, 1410 comprise a plurality of hearing loudness threshold levels at respective test frequencies which the test subject detects in response to hearing audio stimuli at those test frequencies;
wherein hearing correction system's corrected audio output (e.g., 1350, 1450) is generated from corrected loudness values at selected correction frequency sub-bands (“correction EQ frequency bands”);
and the loudspeaker system is driven by the corrected audio output signal which is adjusted for playback in the specific user's automotive interior space at a selected position (e.g., the driver's seat).

Embodiment 3. The hearing correction loudspeaker system of Embodiments 1 or 2, wherein said system further comprises and is responsive to a non-transitory computer-readable storage medium; and program code embodied on the computer-readable storage medium, the program code executable by a processor to determine corrected audio output from administered hearing test results, the administered hearing test results comprising a plurality of hearing loudness threshold levels at respective frequency sub-bands of a bandwidth detected in response to audio stimuli, the corrected audio output comprising corrected loudness values at the respective frequency sub-bands.

Embodiment 4. The hearing correction loudspeaker system of Embodiment 3, further comprising, in said signal processing for generating said corrected audio output signal adjustment one or more dedicated multi-stage compression (DRC+limiter) modules for each frequency sub-band into which the program material is divided.

Embodiment 5. A method for correcting audio for playback via loudspeakers in a room for a specific user, comprising:
(a) providing loudspeaker system configured for use in a listening space (e.g., as illustrated in FIG. 7), said loudspeaker system being responsive to a non-transitory computer-readable storage medium; and program code embodied on the computer-readable storage medium, the program code executable by a processor to determine corrected audio output from administered hearing test results, the administered hearing test results comprising a plurality of hearing loudness threshold levels at respective frequency sub-bands of a bandwidth detected in response to audio stimuli, the corrected audio output comprising corrected loudness values at the respective frequency sub-bands;
(b) administering a hearing test for the specific user to generate hearing test results;
(c) determining corrected audio output signal parameters from the administered hearing test results, the administered hearing test results comprising a plurality of hearing loudness threshold levels at selected audiometry test frequencies, said thresholds being detected in response to audio stimuli at said selected audiometry test frequencies, the corrected audio output comprising corrected loudness values at a selected plurality (e.g., 3-60) EQ frequency sub-bands.

Embodiment 6. A method for correcting a test-subject's loudspeaker's playback to compensate for said test-subject's unique hearing acuity, comprising:
(a) measuring the test subject's hearing acuity by exposing the user to a first plurality (e.g., six) of audiometry test signals or test tones (e.g., 250 Hz, 500 Hz, 1 kHz, 2 kHz, 4 kHz, and 8 kHz) and generating an audiometry plot or profile (e.g., like FIG. 8) for the test subject over a selected frequency range; alternative language: The administered hearing test results comprise a plurality of hearing loudness threshold levels at respective test frequencies which the test subject detects in response to hearing audio stimuli at those test frequencies; The hearing correction system's corrected audio output is generated from corrected loudness values at selected correction frequency sub-bands (“correction EQ frequency bands”);
(b) from the user's hearing (e.g., audiometry) profile, computing or deriving signal processing parameters to generate a corrected audio output signal adjustment comprising a selected plurality of (e.g., 3 to 60) correction EQ signals each covering respective frequency sub-bands (“correction EQ frequency bands”), wherein said EQ correction signals simultaneously generated in said plurality of correction EQ frequency bands effectively represent an inversion of the test subject's audiogram, with surprisingly effective enhancements derived from computation (Table 1) of:
(b1) the Hearing Loss (“HL”) composite at each specific audiometry test frequency from the HL audiometry measurements for each ear, to determine which of the test subject's ears is the “better ear” (L or R) at each frequency (e.g., 250 Hz, 500 Hz, 1 kHz, 2 kHz, 4 kHz, and 8 kHz),
(b2) raw correction signal level adjustment (+ or −) for each of the audiometry test tones or audiometry frequency bands (e.g., 250 Hz, 500 Hz, 1 kHz, 2 kHz, 4 kHz, and 8 kHz),
(b3) correction signal level based on the “better ear” audiometry test results for each of the audiometry test tones or audiometry frequency bands,
(b3) and then, HL with correction for each ear for the audiometry test tones or audiometry frequency bands (e.g., 250 Hz, 500 Hz, 1 kHz, 2 kHz, 4 kHz, and 8 kHz).

Embodiment 7. A hearing correction loudspeaker system, comprising:
one or more digital signal processing (DSP) modules configured to collectively determine corrected audio output for a particular subject or user, said corrected output being derived from age and gender specific predicted hearing loss (e.g., 2110, 2210), the age and gender specific predicted hearing loss comprising a plurality of hearing loudness threshold levels at respective frequencies which the subject or user is predicted to detects in response to hearing audio stimuli at those test frequencies; wherein said hearing correction system's corrected audio output (e.g., 2350, 2450) is generated from corrected loudness values at selected correction frequency sub-bands (“correction EQ frequency bands”).

Embodiment 8. A hearing correction loudspeaker system, comprising:

A loudspeaker system configured for use in a listening space, said loudspeaker system being responsive to a specific user's hearing and a hearing correction system includes signal processing which implements compensatory or corrective signal processing are provided to address the user's hearing deficiencies;
wherein said signal processing is programmed with appropriate signal processing parameters which are derived and implemented to generate a corrected audio output signal adjustment (e.g., 1000, 1360, 1450 comprising a selected plurality of correction EQ signals each covering respective frequency sub-bands (“correction EQ frequency bands”);
wherein administered hearing test results 100, 1310, 1410 comprise a plurality of hearing loudness threshold levels at respective test frequencies which the test subject detects in response to hearing audio stimuli at those test frequencies;
wherein hearing correction system's corrected audio output (e.g., 1350, 1450) is generated from corrected loudness values at selected correction frequency sub-bands (“correction EQ frequency bands”);
and wherein the loudspeaker system is driven by the corrected audio output signal which is adjusted for playback in the specific user's listening space (e.g., in a home or office); and (optionally) wherein said corrected audio output signal adjustment's selected plurality of correction EQ signals are each subject to dedicated compression module for each of said respective frequency sub-bands (“correction EQ frequency bands”).

Embodiment 9. The hearing correction loudspeaker system of Embodiment 7, wherein said hearing correction is implemented in DSP modules, and one or more of said DSP modules are configured to apply first and second parameters to the raw corrections of the frequency sub-bands, the first parameter setting a net correction for a frequency sub-band to zero if the lower hearing loss value at said frequency sub-band is less than the nominal average, the second parameter setting the net correction for the frequency sub-band to a prescribed limit if the raw correction exceeds the prescribed limit (Rules 1 and 2).

Embodiment 10. A hearing correction loudspeaker system, comprising:
one or more digital signal processing (DSP) modules configured to collectively determine corrected audio output for a particular subject or user, said corrected output being derived from administered hearing test results, the administered hearing test results comprising a plurality of hearing loudness threshold levels at respective frequency sub-bands of a bandwidth detected in response to audio stimuli, the corrected audio output comprising corrected loudness values at the respective frequency sub-bands.

Embodiment 11. A hearing correction loudspeaker system, comprising:
one or more transducers or loudspeakers configured to administer audio stimuli to a subject in a dual-monaural manner to generate hearing test results, the hearing test results comprising a plurality of hearing loudness threshold levels at respective frequency sub-bands of a bandwidth encompassed by the audio stimuli; and
one or more digital signal processing (DSP) modules configured to collectively determine corrected audio output from the hearing test results, the corrected audio output comprising corrected loudness values at the respective frequency sub-bands.

Embodiment 12. A hearing correction loudspeaker system, comprising:
one or more transducers or loudspeakers configured to administer audio stimuli to a subject in an ipsilateral manner to generate hearing test results, the hearing test results comprising a plurality of hearing loudness threshold levels at respective frequency sub-bands of a bandwidth encompassed by the audio stimuli; and
one or more digital signal processing (DSP) modules configured to collectively determine corrected audio output from the hearing test results, the corrected audio output comprising corrected loudness values at corresponding respective frequency sub-bands for that subject.

Embodiment 13. A hearing correction loudspeaker system, comprising:
one or more loudspeakers configured to administer audio stimuli to a subject in a binaural manner to generate hearing test results, the hearing test results comprising a plurality of hearing loudness threshold levels at respective frequency sub-bands of a bandwidth encompassed by the audio stimuli; and
one or more digital signal processing (DSP) modules configured to collectively determine corrected audio output from the hearing test results, the corrected audio output comprising corrected loudness values at the respective frequency sub-bands for that subject.

Embodiment 14. A hearing correction loudspeaker system, comprising:
one or more loudspeakers configured to administer audio stimuli to a subject to generate hearing test results, one or more loudspeakers being arranged as intended for use in an acoustic space to emit the audio stimuli, the hearing test results comprising a plurality of hearing loudness threshold levels at respective frequency sub-bands of a bandwidth encompassed by the audio stimuli; and
one or more digital signal processing (DSP) modules configured to collectively determine corrected audio output from the hearing test results, the corrected audio output comprising corrected loudness values at the respective frequency sub-bands.

Embodiment 15. A hearing correction loudspeaker system, comprising:
one or more transducers (e.g., headphones IEMs or earbuds) configured to administer audio stimuli to a subject to generate hearing test results, the hearing test results comprising a plurality of hearing loudness threshold levels at respective frequency sub-bands of a bandwidth encompassed by the audio stimuli; and
one or more digital signal processing (DSP) modules configured to collectively determine corrected audio output from the hearing test results, the corrected audio output comprising corrected loudness values at the respective frequency sub-bands,
wherein the hearing test results are derived by inducing, acquiring, and interpreting otoacoustic emissions.

Embodiment 16. A hearing correction loudspeaker system, comprising:
one or more loudspeakers configured to administer audio stimuli to a subject to generate hearing test results, the hearing test results comprising a plurality of hearing loudness threshold levels at respective frequency sub-bands of a bandwidth encompassed by the audio stimuli; and
one or more digital signal processing (DSP) modules configured to collectively determine corrected audio output from the hearing test results, the corrected audio output comprising corrected loudness values at the respective frequency sub-bands,
wherein the one or more DSP modules comprise an input module configured to receive an audiogram comprising the administered hearing test results.

Embodiment 17. A hearing correction loudspeaker system, comprising:
one or more loudspeakers configured to administer audio stimuli to a subject to generate hearing test results, the hearing test results comprising a plurality of hearing loudness threshold levels at respective frequency sub-bands of a bandwidth encompassed by the audio stimuli; and
one or more digital signal processing (DSP) modules configured to collectively determine corrected audio output from the hearing test results, the corrected audio output comprising corrected loudness values at the respective frequency sub-bands,
wherein the one or more DSP modules comprise a crossover filter module configured to subdivide the administered hearing test results into the respective frequency sub-bands over the bandwidth.

Embodiment 18. A hearing correction loudspeaker system, comprising:
one or more loudspeakers configured to administer audio stimuli to a subject to generate hearing test results, the hearing test results comprising a plurality of hearing loudness threshold levels at respective frequency sub-bands of a bandwidth encompassed by the audio stimuli; and
one or more digital signal processing (DSP) modules configured to collectively determine corrected audio output from the hearing test results, the corrected audio output comprising corrected loudness values at the respective frequency sub-bands,
wherein the one or more DSP modules further comprise a plurality of dynamic range compression (DRC) modules, the DRC modules each operating at discrete frequency sub-bands from one another . . . Note: “plurality” covers multiple compressors (at least one for each frequency sub-band) and also includes optional multi-stage compression for each frequency sub-band.

Embodiment 19. A hearing correction loudspeaker system, comprising:
one or more loudspeakers configured to administer audio stimuli to a subject to generate hearing test results, the hearing test results comprising a plurality of hearing loudness threshold levels at respective frequency sub-bands of a bandwidth encompassed by the audio stimuli; and
one or more digital signal processing (DSP) modules configured to collectively determine corrected audio output from the hearing test results, the corrected audio output comprising corrected loudness values at the respective frequency sub-bands,
wherein the one or more DSP modules further comprise a mixer module configured to generate or construct a corrected audio output based upon output of the DRC modules.

Embodiment 20. A hearing correction loudspeaker system, comprising:
one or more loudspeakers configured to administer audio stimuli to a subject to generate hearing test results, the hearing test results comprising a plurality of hearing loudness threshold levels at respective frequency sub-bands of a bandwidth encompassed by the audio stimuli; and
one or more digital signal processing (DSP) modules configured to collectively determine corrected audio output from the hearing test results, the corrected audio output comprising corrected loudness values at each of a plurality of frequency sub-bands,
wherein the hearing loudness threshold levels and the corrected loudness values represent hearing loss values.

Embodiment 21. A hearing correction loudspeaker system, comprising:
one or more loudspeakers configured to administer audio stimuli to a subject to generate hearing test results, the hearing test results comprising a plurality of hearing loudness threshold levels at respective frequency sub-bands of a bandwidth encompassed by the audio stimuli; and
one or more digital signal processing (DSP) modules configured to collectively determine corrected audio output from the hearing test results, the corrected audio output comprising corrected loudness values at the respective frequency sub-bands,
wherein the one or more DSP modules are configured to calculate a composite better-ear average based upon lower hearing loss values for either ear at each of the frequency sub-bands . . . Note—the term “frequency sub-bands” normally means the passbands into which the audio spectrum is divided for purposes of correction (low/med/high up to dozens of narrow bands, in principle); but here, it may also refer to the individual test frequencies of an audiogram.

Embodiment 22. The hearing correction loudspeaker system of Embodiment 21, wherein the one or more DSP modules are configured to apply a parameter to the raw corrections of the frequency sub-bands, the parameter setting a net correction for a frequency sub-band to zero if the lower hearing loss value at said frequency sub-band is less than the composite better-ear average.

Embodiment 23. The hearing correction loudspeaker system of Embodiment 21, wherein the one or more DSP modules are configured to apply a parameter to the raw corrections of the frequency sub-bands, the parameter setting a net correction for a frequency sub-band to a prescribed limit if the raw correction exceeds the prescribed limit.

Embodiment 24. The hearing correction loudspeaker system of Embodiment 21, wherein the one or more DSP modules are configured to apply first and second parameters to the raw corrections of the frequency sub-bands, the first parameter setting a net correction for a frequency sub-band to zero if the lower hearing loss value at said frequency sub-band is less than the composite better-ear average, the second parameter setting the net correction for the frequency sub-band to a prescribed limit if the raw correction exceeds the prescribed limit.

Embodiment 25. A hearing correction loudspeaker system, comprising:
one or more loudspeakers configured to administer audio stimuli to a subject to generate hearing test results, the hearing test results comprising a plurality of hearing loudness threshold levels at respective frequency sub-bands of a bandwidth encompassed by the audio stimuli; and
one or more digital signal processing (DSP) modules configured to collectively determine corrected audio output from the hearing test results, the corrected audio output comprising corrected loudness values at the respective frequency sub-bands, wherein the one or more DSP modules are configured to calculate a nominal average based upon hearing loss values for both ears at each of the frequency sub-bands.

Embodiment 26. The hearing correction loudspeaker system of Embodiment 25, wherein the one or more DSP modules are configured to calculate raw corrections at the frequency sub-bands, the raw corrections being based upon a calculation involving a lower hearing loss value at said frequency sub-band and the nominal average.

Embodiment 27. The hearing correction loudspeaker system of Embodiment 26, wherein the one or more DSP modules are configured to apply a parameter to the raw corrections of the frequency sub-bands, the parameter setting a net correction for a frequency sub-band to zero if the lower hearing loss value at said frequency sub-band is less than the nominal average (Rule 1).

Embodiment 28. The hearing correction loudspeaker system of Embodiment 26, wherein the one or more DSP modules are configured to apply a parameter to the raw corrections of the frequency sub-bands, the parameter setting a net correction for a frequency sub-band to a prescribed limit if the raw correction exceeds the prescribed limit (Rule 2).

Embodiment 29. The hearing correction loudspeaker system of Embodiment 25, wherein the one or more DSP modules are configured to apply first and second parameters to the raw corrections of the frequency sub-bands, the first parameter setting a net correction for a frequency sub-band to zero if the lower hearing loss value at said frequency sub-band is less than the nominal average, the second parameter setting the net correction for the frequency sub-band to a prescribed limit if the raw correction exceeds the prescribed limit (Rules 1 and 2).

Embodiment 30. A hearing correction loudspeaker system, comprising any combination or combinations of the hearing correction loudspeaker systems of Embodiments 1 to 30.

The following is claimed:

1. A hearing correction loudspeaker system comprising:
one or more loudspeakers; and
a hearing correction system comprising signal processing elements configured to implements corrective signal processing to at least partially compensate for hearing deficiencies of a user based on hearing profile information for the user;
wherein the system is configured to receive input audio;
wherein the signal processing elements are configured to generate corrected audio output signals based on the input audio and the hearing profile information, wherein the corrected audio output signals are configured to at least partially compensate for hearing deficiencies of the user; and
wherein the system is configured to drive the one or more loudspeakers by the corrected audio output signals to produce audio playback that is corrected to at least partially compensate for hearing deficiencies of the user.

2. The hearing correction loudspeaker system of claim 1, wherein the hearing profile information is based at least on an age and/or gender of the listener.

3. The hearing correction loudspeaker system of claim 1, wherein the hearing profile information is based on otoacoustic emissions.

4. The hearing correction loudspeaker system of claim 1, wherein the hearing profile information is based on an audiogram specific to the user.

5. The hearing correction loudspeaker system of claim 1, wherein the hearing profile information comprises information corresponding to a plurality of hearing loudness threshold levels at respective test frequencies which the user detected in response to hearing audio stimuli at those test frequencies.

6. The hearing correction loudspeaker system of claim 5, wherein the corrected audio output signals are generated from corrected loudness values at correction frequency sub-bands.

7. The hearing correction loudspeaker system of claim 5, wherein the signal processing elements implement dedicated compression modules for each of the respective frequency sub-bands.

8. The hearing correction loudspeaker system of claim 1, wherein the hearing profile information comprises composite “better ear” information for each of a plurality of hearing loudness threshold levels at respective test frequencies which the user detected in response to hearing audio stimuli at those test frequencies.

9. The hearing correction loudspeaker system of claim 1, wherein:
the hearing profile information includes right-ear hearing profile information and left-ear hearing profile information;
the signal processing elements are configured to generate right-ear corrected audio output signals based on the input audio and the right-ear hearing profile information;
the signal processing elements are configured to generate left-ear corrected audio output signals based on the input audio and the left-ear hearing profile information; and
the system is configured to drive the one or more loudspeakers to use beamforming provide a right audio zone at a right ear of the user to present right-ear-corrected audio that is corrected to at least partially compensate for hearing deficiencies of the right ear of the user, and to use beamforming provide a left audio zone at a left ear of the user to present left-ear-corrected audio that is corrected to at least partially compensate for hearing deficiencies of the left ear of the user.

10. The hearing correction loudspeaker system of claim 1, wherein the system is configured to use the one or more loudspeakers to output test tones for conducting an audiogram hearing test.

11. An audio system comprising:
one or more loudspeakers; and
one or more hardware processors configured to:
access hearing profile information;
access audio content;
determine modified audio signals based at least in part on the audio content and the hearing profile information; and
drive the one or more loudspeakers based on the modified audio signals.

12. The audio system of claim 11, wherein the hearing profile information is based at least on an age and/or gender of the listener.

13. The audio system of claim 11, wherein the hearing profile information is based on otoacoustic emissions.

14. The audio system of claim 11, wherein the hearing profile information is based on an audiogram specific to the user.

15. The audio system of claim 11, wherein the hearing profile information comprises information corresponding to a plurality of hearing loudness threshold levels at respective test frequencies which the user detected in response to hearing audio stimuli at those test frequencies.

16. The audio system of claim 15, wherein the corrected audio output signals are generated from corrected loudness values at correction frequency sub-bands.

17. The audio system of claim 15, wherein the signal processing elements implement dedicated compression modules for each of the respective frequency sub-bands.

18. The audio system of claim 11, wherein the hearing profile information comprises composite “better ear” information for each of a plurality of hearing loudness threshold levels at respective test frequencies which the user detected in response to hearing audio stimuli at those test frequencies.

19. The audio system of claim 11, wherein:
the hearing profile information includes right-ear hearing profile information and left-ear hearing profile information;
the signal processing elements are configured to generate right-ear corrected audio output signals based on the input audio and the right-ear hearing profile information;
the signal processing elements are configured to generate left-ear corrected audio output signals based on the input audio and the left-ear hearing profile information; and
the system is configured to drive the one or more loudspeakers to use beamforming provide a right audio zone at a right ear of the user to present right-ear-corrected audio that is corrected to at least partially compensate for hearing deficiencies of the right ear of the user, and to use beamforming provide a left audio zone at a left ear of the user to present left-ear-corrected audio that is corrected to at least partially compensate for hearing deficiencies of the left ear of the user.

20. The audio system of claim 11, wherein the system is configured to use the one or more loudspeakers to output test tones for conducting an audiogram hearing test.
